(19) United States
(12) Patent Application Publication
(10) Pub. No.: US 2025/0064852 A1
(43) Pub. Date: Feb. 27, 2025

(54) CD5 Modified Cells Comprising Chimeric Antigen Receptors (CARs) for Treatment of Solid Tumors

(71) Applicant: The Trustees of the University of Pennsylvania, Philadelphia, PA (US)

(72) Inventor: Marco Ruella, Philadelphia, PA (US)

(21) Appl. No.: 18/719,050

(22) PCT Filed: Dec. 13, 2022
(86) PCT No.: PCT/US2022/081463

§ 371 Date: Jun. 12, 2024

Related U.S. Application Data
(60) Provisional application No. 63/380,012, filed on Oct. 18, 2022.
(60) Provisional application No. 63/289,364, filed on Dec. 14, 2021.

(51) Int. Cl. A61K 35/17; A61K 39/00; A61K 48/00; A61P 35/00; C12N 9/22; C12N 15/11; C12N 15/86

(52) U.S. Cl. CPC A61K 35/17; A61K 39/4611; A61K 39/4631; A61K 39/464468; A61K 48/005; A61P 35/00; C12N 9/22; C12N 15/111; C12N 15/86; A61K 2239/13; A61K 2239/21; A61K 2239/22; A61K 2239/38; C12N 2310/20; C12N 2740/15041

(57) ABSTRACT

The present disclosure provided compositions and methods for treating solid tumors. In certain aspects, the disclosure provides modified immune cells (i.e. CAR T cells) wherein CD5 has been disrupted or knocked-out.

### CROSS-REFERENCE TO RELATED APPLICATIONS

The present application is a 35 U.S.C. § 371 national phase application from, and claims priority to, International Application No. PCT/US2022/081463 filed Dec. 13, 2022, which claims priority under 35 U.S.C. § 119(e) U.S. Provisional Application No. 63/289,364, filed Dec. 14, 2021 and U.S. Provisional Application No. 63/380,012, filed Oct. 18, 2022, each of which are hereby incorporated by reference in its entirety.

### REFERENCE TO AN ELECTRONIC SEQUENCE LISTING

The Sequence Listing submitted herewith as a xml file named “046483-7358WO1(03082) Sequence Listing,” created on Dec. 13, 2022 and having a size of 87,592 bytes bytes, is incorporated herein by reference in its entirety.

### BACKGROUND OF THE INVENTION

A need exists for improved compositions and methods for treating solid tumors. The present embodiments addresses this need as well as others.

### SUMMARY OF THE INVENTION

As described herein, the present invention relates to compositions and methods for treating solid tumors.

In one aspect, the invention provides a method of treating a solid tumor in a subject in need thereof. The method comprises administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen.

In another aspect, the invention provides a method of treating a solid tumor in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, and wherein the growth of the tumor is inhibited for at least 10, 20, 30, 40, 50, 60, 70, 80, 90, 100, 120, 150, 200, 240 days.

In another aspect, the invention provides a method of treating a solid tumor with enhanced efficacy in a subject in need thereof. The method comprises administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, and wherein the efficacy of the population of immune cells comprising the mutated endogenous CD5 gene is greater than a population of immune cells comprising a non-mutated endogenous CD5 gene.

In various embodiments of the above aspects or any other aspect of the invention delineated herein, the pharmaceutical composition comprises about 1×10⁶ to about 1×10⁹, about 1×10⁷ to about 1×10⁹, about 1×10⁸ to about 1×10⁹, about 2×10⁸ to about 1×10⁹, about 3×10⁸ to about 1×10⁹, about 3×10⁸ to about 1×10⁹, about 3×10⁸ to about 1×10⁹, about 4×10⁸ to about 1×10⁹, about 5×10⁸ to about 1×10⁹, about 6×10⁸, about 7×10⁸ to about 1×10⁹, about 8×10⁸, about 9×10⁸ to about 1×10⁹ of the immune cells.

In certain embodiments, at least 75%, 80%, 85%, 90%, 91%, 92%, 93%, 94%, 95%, 96%, 97%, 98%, 99%, or 100% of the population of immune cells comprise the mutated endogenous CD5 gene.

In certain embodiments, at least 50%, 55%, 60%, 65%, 75%, 80%, 85%, 90%, 91%, 92%, 93%, 94%, 95%, 96%, 97%, 98%, 99%, or 100% of the population of immune cells comprise the heterologous chimeric antigen receptor (CAR).

In certain embodiments, the immune cells do not express or comprise an endogenous full length CD5 protein. In certain embodiments, the mutated CD5 gene has an exon 1 or exon 2 mutation, insertion or deletion. In certain embodiments, the exon 1 or exon 2 mutation, insertion or deletion inhibits or reduces the expression of the endogenous full length CD5 protein.

In certain embodiments, the endogenous CD5 gene comprises a gene edited mutation, insertion or deletion. In certain embodiments, the gene edited mutation, insertion or deletion is a nuclease mediated gene mutation, insertion or deletion. In certain embodiments, the nuclease mediated gene mutation, insertion or deletion targets exon 1 or exon 2 of the CD5 gene. In certain embodiments, the gene edited or nuclease mediated gene mutation, insertion or deletion utilizes an sgRNA comprising the nucleotide sequence of SEQ ID NO: 45.

In certain embodiments, the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In certain embodiments, the modified immune cell is a T cell.

In certain embodiments, the antigen binding domain of the CAR comprises a complementarity determining region (CDR) comprising an amino acid sequence selected from the group consisting of SEQ ID NOs: 27-32 and 39-44.

In certain embodiments, the antigen binding domain of the CAR comprises a heavy chain variable region comprising the amino acid sequence selected from the group consisting of SEQ ID NOs: 19, 25, and 37 and/or a light chain variable region comprising the amino acid sequence selected from the group consisting of SEQ ID NOs: 20, 26, and 38.

In certain embodiments, the antigen binding domain of the CAR comprises an scFv comprising the amino acid sequence selected from the group consisting of SEQ ID NOs: 17, 18, 23, 24, 35, or 36.

In certain embodiments, the CAR comprises an amino acid sequence selected from the group consisting of SEQ ID NOs: 15, 16, 21, 22, 33, and 34. In certain embodiments, the CAR is encoded by a nucleic acid sequence selected from the group consisting of SEQ ID NOs: 1-6.

In certain embodiments, the solid tumor is prostate cancer (e.g., pancreatic ductal adenocarcinoma (“PDAC”), fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

Another aspect of the invention includes a method of treating a solid tumor in a subject in need thereof. The method comprises administering to the subject a vector comprising a targeting moiety that binds to a CD5 expressing immune cell and a polynucleotide encoding a gene editing system and a chimeric antigen receptor (CAR). The CAR comprises an antigen binding domain, a transmembrane domain, and an intracellular domain, wherein the antigen binding domain binds to a solid tumor antigen. The gene editing system is configured to modify the endogenous CD5 gene to inhibit, eliminate, or reduce expression of endogenous CD5. The vector transduces a CD5 expressing immune cell in the subject to mutate the endogenous CD5 gene to inhibit or reduce expression of endogenous CD5 and express the CAR in the immune cell with the mutated endogenous CD5 gene.

In certain embodiments, the vector is a viral vector. In certain embodiments, the viral vector is a lentivirus.

In certain embodiments, the targeting moiety is an antibody or other type of targeting moiety (e.g. a scFv, an antigen binding domain, a DARPIN, a VHH, or a FN3 domain).

In certain embodiments, the solid tumor is fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

In certain embodiments, the transduced cells do not express or comprise an endogenous full length CD5 protein. In certain embodiments, the mutated CD5 gene has an exon 1 or exon 2 mutation, insertion or deletion. In certain embodiments, the exon 1 or exon 2 mutation, insertion or deletion inhibits or reduces the expression of the endogenous full length CD5 protein.

In certain embodiments, the endogenous CD5 gene comprises a gene edited (e.g., CRISPR mediated) gene mutation, insertion or deletion. In certain embodiments, the gene edited gene mutation, insertion or deletion is a CRISPR/Cas9 mediated gene mutation, insertion or deletion. In certain embodiments, the gene edited gene mutation, insertion or deletion targets exon 1 or exon 2 of the CD5 gene. In certain embodiments, the gene edited (e.g., CRISPR/Cas9 mediated) gene mutation, insertion or deletion utilizes an sgRNA comprising the nucleotide sequence of SEQ ID NO: 45.

In certain embodiments, the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), 3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, pi85erbB2, pi80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In certain embodiments, the modified immune cell is a T cell.

In certain embodiments, the antigen binding domain of the CAR comprises a complementarity determining region (CDR) comprising an amino acid sequence selected from the group consisting of SEQ ID NOs: 27-32 and 39-44.

In certain embodiments, the antigen binding domain of the CAR comprises a heavy chain variable region comprising the amino acid sequence selected from the group consisting of SEQ ID NOs: 19, 25, and 37 and/or a light chain variable region comprising the amino acid sequence selected from the group consisting of SEQ ID NOs: 20, 26, and 38. In certain embodiments, the antigen binding domain of the CAR comprises an scFv comprising the amino acid sequence selected from the group consisting of SEQ ID NOs: 17, 18, 23, 24, 35, or 36.

In certain embodiments, the CAR comprises an amino acid sequence selected from the group consisting of SEQ ID NOs: 15, 16, 21, 22, 33, and 34.

In certain embodiments, the CAR is encoded by a nucleic acid sequence selected from the group consisting of SEQ ID NOs: 1-6.

Another aspect of the invention provides a modified immune cell comprising a chimeric antigen receptor (CAR), wherein the CAR comprises an antigen binding domain, a transmembrane domain, and an intracellular domain, and wherein the endogenous CD5 gene has been mutated, modified, disrupted, or knocked-out, and wherein the antigen binding domain binds to a solid tumor antigen.

In certain embodiments, the antigen binding domain of the CAR binds to mesothelin, CD5, CD19, CD2, CD7, a tumor-specific antigen (TSA), a tumor associated antigen (TAA), a glioma-associated antigen, carcinoembryonic antigen (CEA), 3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Another aspect of the invention includes a composition (e.g., a pharmaceutical composition) comprising a population of any of the modified immune cells contemplated herein.

In certain embodiments, at least 75%, 80%, 85%, 90%, 91%, 92%, 93%, 94%, 95%, 96%, 97%, 98%, 99%, or 100% of the population of the modified immune cells comprise the mutated endogenous CD5 gene.

In certain embodiments, at least 50%, 55%, 60%, 65%, 75%, 80%, 85%, 90%, 91%, 92%, 93%, 94%, 95%, 96%, 97%, 98%, 99%, or 100% of the population of immune cells comprise the heterologous chimeric antigen receptor (CAR).

Another aspect of the invention includes a method of generating any of the modified immune cells contemplated herein. The method comprises transfecting or transducing the immune cell with a nucleic acid encoding the CAR and/or at least one nucleic acid that knocks-out, mutates, or disrupts endogenous CD5 gene, such as an exon of the CD5 gene, including but not limited to exon 1 or exon 2 of the CD5 gene, wherein the CAR comprises an antigen binding domain, a transmembrane domain, and an intracellular domain, wherein the antigen binding domain binds to a solid tumor antigen.

In certain embodiments, the antigen binding domain binds to mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In certain embodiments, the immune cell is transduced in vivo or ex vivo. In certain embodiments, the immune cell is transduced in vivo.

In certain embodiments, the immune cell is a T cell.

### BRIEF DESCRIPTION OF THE DRAWINGS

The following detailed description of specific embodiments will be better understood when read in conjunction with the appended drawings. For the purpose of illustrating the embodiments, there are shown in the drawings exemplary embodiments. It should be understood, however, that the embodiments are not limited to the precise arrangements or the data of some embodiments shown in the drawings.

FIGS. 1A-1D: In vivo anti-tumor effect of CD5 knock out CAR T cells against solid cancer. FIG. 1A: Immunodeficient (NSG) mice were engrafted with ASPC1 pancreatic cancer cells (luciferase+, subcutaneously). At day 14 mice were randomized based on tumor burden to receive control untransduced T cells (UTD), CD5 knock out (KO) UTD, mock control anti-mesothelin CAR T cells or CD5 KO CAR T cells. Tumor burden was measured over time by caliper and luminescence. Caliper measurement showed complete remission in both groups but only CD5 KO CAR T maintained the remission at long term while control CAR T relapsed at about 3 months. FIG. 1B: Tumor burden by bioluminescence showed early and deep tumor clearance by CD5 KO CAR T as compared to mock control CAR T or UTD. FIG. 1C: Single-mouse tumor burden by caliper over time in the CD5 CAR T group.

FIG. 1D: In vivo expansion in the peripheral blood of T cells was measured in all groups by flow cytometry. CD5 KO CAR T showed higher expansion as compared to mock control CAR T.

FIG. 2A-2E: CD5 KO in CART5 T cells improved engineered T cell product compared to Mock KO CART5. FIG. 2A: CD5 knock out CAR T expansion protocol. FIG. 2B: Left, representative histogram plot showing expression of CD5 and CAR5 on the groups of engineered T cells on Day 8 of expansion. n=4 donors. Right, representative histogram plot showing mean fluorescence intensity (MFI) of CD5 and CAR5 on the same cells. n=4 donors. FIG. 2C: Expansion of bead stimulated T cells shown in population doublings (log 2 transformed fold change of total cell counts). n=4 donors. FIG. 2D: T cell memory phenotypes of each engineered T cell groups at the end of expansion. Tₙₐᵢᵥₑ, naïve T cells, CD45RA⁺CCR7⁺; T_{CM}, central memory T cells, CD45RA⁻CCR7⁺; T_{EM}, effector memory T cells, CD45RA⁻CD27⁻; T_{EMRA}, effector memory T cells re-expressing CD45RA, CD45RA⁺CCR7⁻. n=4 donors. FIG. 2E: Expression of T cell exhaustion markers PD1 and LAG3 on each engineered T cell group at the end of expansion in CD8+ cells. n=4 donors.

FIG. 3A-3C: CD5-targeting sgRNA demonstrated strong efficiency and specificity.

FIG. 3A: Eight CD5-targeting sgRNA sequences were tested. CD5_gRNA_4 exhibited the strongest knockout efficiency and was used for all future experiments. FIG. 3B: CD5 KO by CD5_gRNA_4 was confirmed by flow cytometry as well as SDS-PAGE and immunoblotting. FIG. 3C: iGUIDE-sequencing verified high on-target and low off-target efficiency of CD5_gRNA_4 using both TrueCut Cas9 or SpyFi Cas9. n=2 donors.

FIG. 4A-4B: CD5-targeting CAR T cell exhibited strong specificity for CD5. FIG. 4A: Representative example of specificity screen where Mock KO UTD and CD5 KO CART5 were added to fixed confirmation slides. FIG. 4B: Full library and confirmation screen results concluded that one CAR-specific interaction, the primary target CD5, was identified for CD5 KO CART5 cells. All other interactions were determined as very weak or CAR-independent.

FIG. 5A-5B: Primary human CART5 T cells selectively targeted and killed CD5+ cells. FIG. 5A: Primary human T cells were generated. CD5 and CAR5 expression was monitored and recorded daily for ten days in Mock KO UTD, CD5 KO UTD, Mock KO CART5, and CD5 KO CART5 T cells. n=2 donors. FIG. 5B: Expression of T cell exhaustion markers PD1 and LAG3 on each engineered T cell group at the end of expansion in CD4+ cells. n=4 donors.

FIG. 6A-6E: CD5 KO in CART5 T cells improved engineered T cell product compared to Mock KO CART5. FIG. 6A: Percent cytotoxicity of UTD or CART5 cells against CD5+ primary T-ALL or primary Sezary cells after 48 h. FIG. 6A: Percent cytotoxicity of UTD or CART5 cells against CD5+ primary T-ALL or primary Sezary cells after 48 h. FIG. 6B: Bioluminescence imaging of tumor burden in each NSG mouse (n=5 mice) engrafted with Jurkat T-ALL cell line. Representative data of 3 independent experiments performed with different donor T cells is shown. FIG. 6C: Survival rate of each treatment group in xenograft T-ALL model is shown in a Kaplan-Meier survival curve. FIG. 6D: Left, absolute cell counts of huCD3⁺ T cells in 100 μL of mice blood at Day 15. Right, absolute cell counts of huCD3⁺ T cells in 100 μL of mice blood over time. FIG. 6E: Bioluminescence imaging of tumor burden in each NSG mouse (n=6 mice) engrafted with primary T-ALL cells.

FIG. 7A-7B: Depletion of CD5 enhanced CAR therapy in solid tumor models. FIG. 7A: Bioluminescence imaging of tumor burden in each NSG mouse (n=7 mice) engrafted with AsPC1 PDAC cell line. FIG. 7B: Absolute cell counts of huCD45⁺ T cells in 100 μL of mice blood at Day 58.

FIG. 8A-8G: CD5 deletion enhanced CAR signaling via phosphorylation and calcium influx. FIG. 8A: Schematic describing the inhibitory role of CD5 in T cell activation: upon activation, CD5 recruited several mediators to the cell membrane including SHP-1, CBL, and CBL-B. CBL ubiquitinates and promoted the degradation of PLC71, leading to reduction of total protein levels. SHP-1 dephosphorylated LAT, an upstream positive regulator of PLC71, while CBL-B ubiquitinates and promoted its degradation. FIG. 8B: Quantitative measurement of phosphorylated proteins on CD5 KO T cells compared to Mock KO T cells.

FIG. 8C: Total PLC71 and 3-actin expression in primary Mock KO or CD5 KO T cells activated via TCR by CD3/CD28 bead stimulation for 45 minutes. FIG. 8D: Calcium flux in Mock KO or CD5 KO T cells was measured in real time by flow cytometry. FIG. 8E: Principal component analysis showed distinct grouping of Mock KO and CD5 KO CART5 cells. n=2 donors. FIG. 8F: Gene set enrichment analysis identified calcium dependent events and DAG and IP3 signaling as enriched pathways within CD5 KO cells. FIG. 8G: Heat map of Z-scores of genes associated to calcium dependent events and DAG and IP3 signaling.

FIG. 9A-91: Depletion of CD5 enhanced CAR therapy in several tumor models: FIG. 9A: Tumor volume (mm³) in each NSG mouse (n=7 mice) engrafted with AsPC1 PDAC cell line. FIG. 9B: Bioluminescence imaging of tumor burden in each NSG mouse. FIG. 9C: Absolute cell counts of huCD45⁺ T cells in 100 μL of mice blood at Day 58. FIG. 9D: Tumor volume (mm³) in each NSG mouse (n=4-5 mice) engrafted with AsPC1 PDAC cell line.

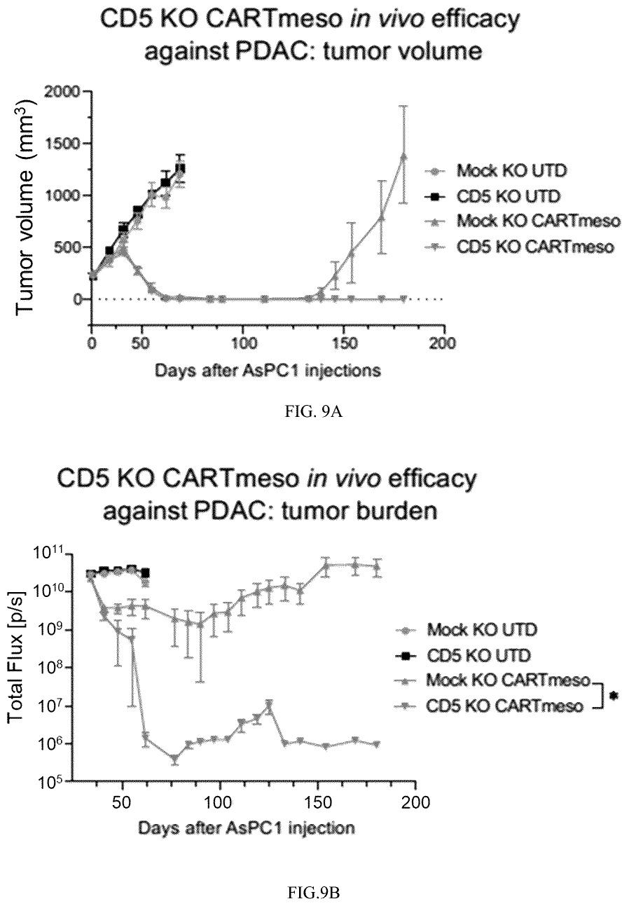

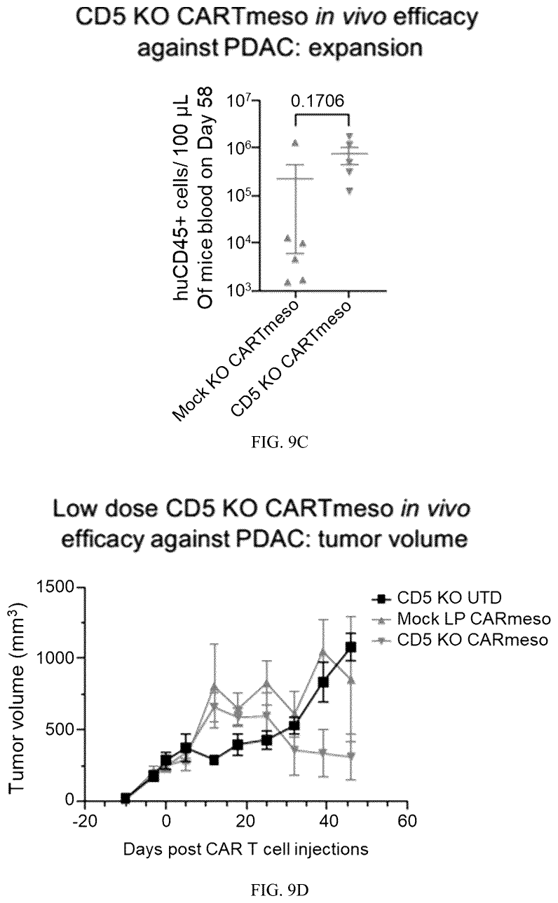

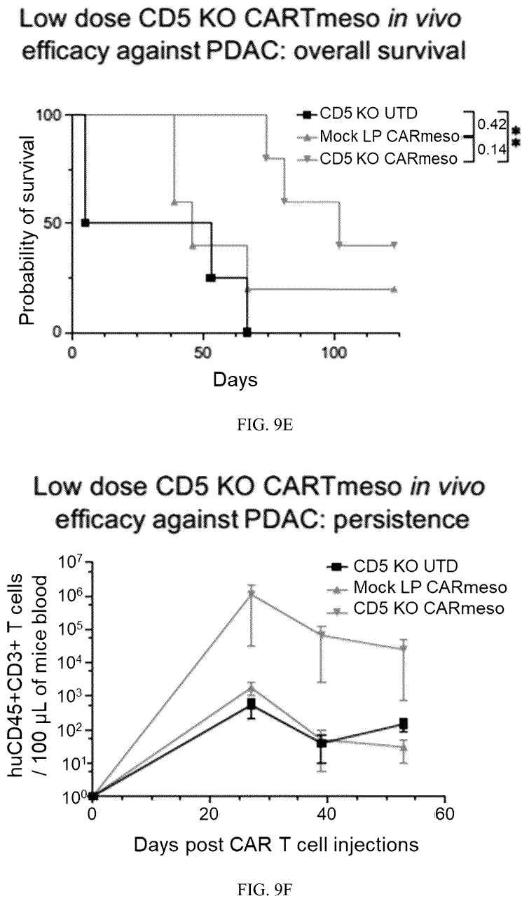

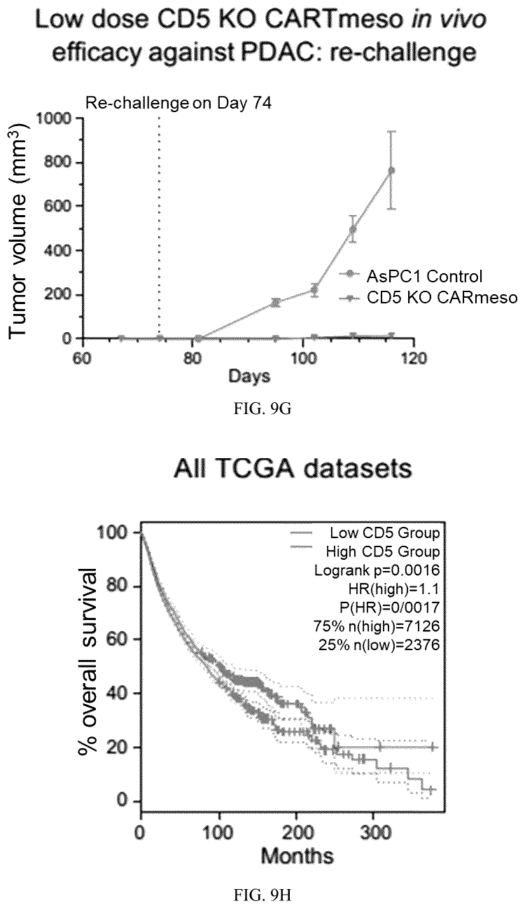

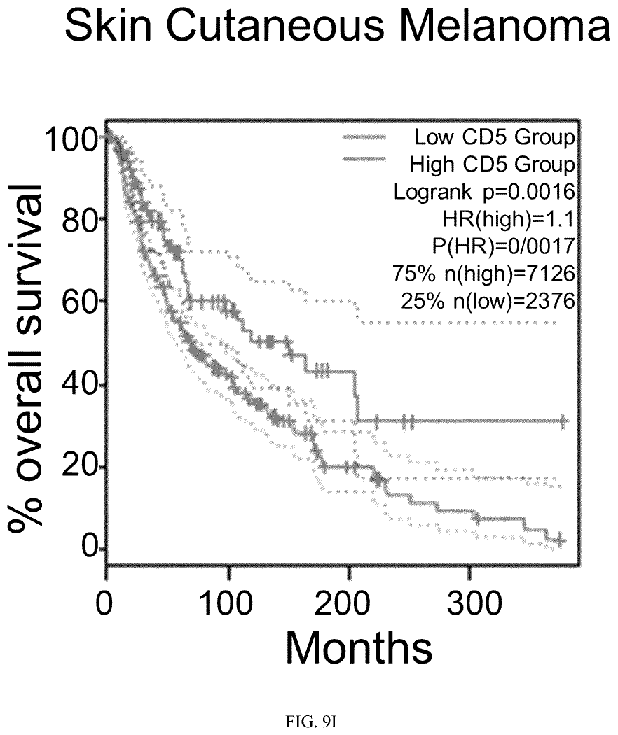

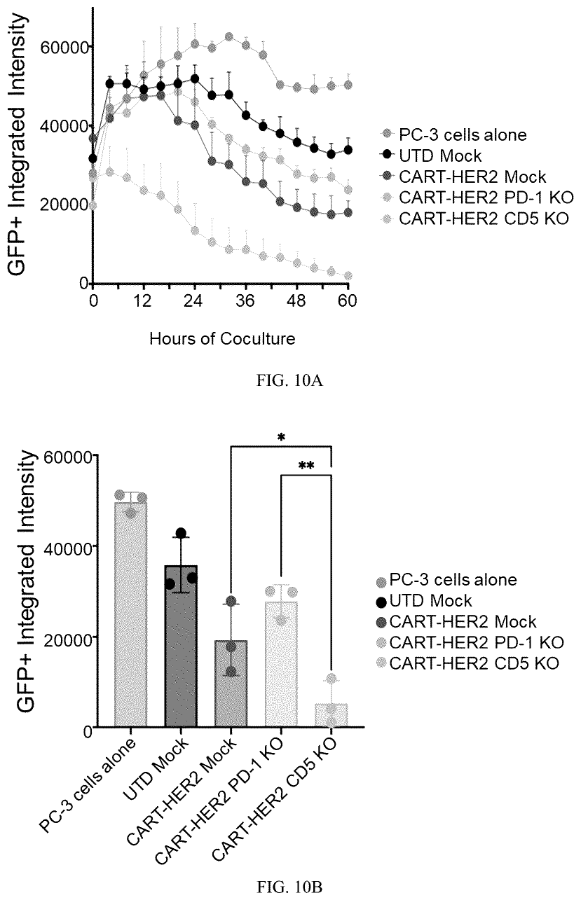

Representative data of 2 independent experiments is shown. FIG. 9E: Survival rate of each treatment group in xenograft AsPC1 model is shown in Kaplan-Meier survival curve. FIG. 9F: Absolute cell counts of huCD3⁺huCD45⁺ T cells in 100 μL of mice blood over time. FIG. 9G: Tumor volume (mm³) in control NSG mice or mice treated with CD5 KO CARmeso and re-challenged with AsPC1 cells on Day 74. FIG. 9H: All patients were divided in two groups based on high and low CD5 expression (75% high; 25% low) within TCGA GEPIA2 database. CD5 expression was normalized by CD3F to account for tumor-infiltrating lymphocyte T cell numbers. FIG. 9I: Skin cutaneous melanoma patients were divided in two groups based on high and low CD5 expression (75% high; 25% low). CD5 expression was normalized by CD3F to account for tumor-infiltrating lymphocyte T cell numbers.

FIG. 10A-10B: Anti-HER2 CAR T cells (Clone 4D5) were generated following the manufacturing timeline previously described. FIG. 10A: As a model to test the efficacy of BTLA KO on anti-HER2 CAR T cells, PC3, a HER2+ prostate adenocarcinoma, was used. 4,000 PC3 cells were plated in a 96-well plate 24 hours prior to addition of 1000 HER2+ CAR T cells or controls. GFP+ (PC3) intensity was monitored every 3 hours using the Incucyte® Live-Cell Analysis System. FIG. 10B: Killing is shown at the 48 hour timepoint, demonstrating significantly higher cytotoxicity in CD5 KO over wild type anti-HER2 CAR.

### DETAILED DESCRIPTION

The present disclosure provides compositions and methods for treating solid tumors using chimeric antigen receptor (CAR) T cells wherein the endogenous CD5 gene of the T cell is mutated, such that, for example, the endogenous CD5 protein expression or function is inhibited or reduced, completely or partially. In some embodiments, the CD5 gene is said to be knocked out such that there is no endogenous CD5 protein expression or a functional CD5 protein is not expressed.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting. Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably +5%, even more preferably +1%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present embodiments include, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of,” “consists,” or “consisting essentially of.”

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an NMC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” as it is used in reference to a specific gene, refers to the gene that is naturally occurring in the organism, cell, tissue or system without the introduction of an exogenous or heterologous substance, such a nucleic acid molecule. For example, an “an endogenous CD5 gene” refers to the gene encoding the CD5 protein that is naturally occurring in the cell. “Endogenous” in reference to other materials, means that such material is from or produced inside an organism, cell, tissue or system without any exogenous material being introduced into the organism, cell, tissue, or system.

As used herein, the term “exogenous” refers to any material introduced from or produced outside an organism, cell, tissue or system. For example, a chimeric antigen receptor can be produced in a cell by the introduction of an exogenous nucleic acid molecule encoding the chimeric antigen receptor. A nucleic acid molecule that is introduced into the cell can also be referred to as a “heterologous” nucleic acid molecule.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence. The term “expression” as it is made in reference to a protein means the amounts of the protein that is present or made in a cell, organism, or system.

“Expression vector” refers to a vector comprising a recombinant polynucleotide comprising expression control sequences operatively linked to a nucleotide sequence to be expressed. An expression vector can comprise sufficient cis-acting elements for expression. In some embodiments, other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” or “therapeutic amount” is indicated, the precise amount of the compositions to be administered can be determined by a physician or researcher with consideration of individual differences in age, weight, tumor size, extent of infection or metastasis, and condition of the patient (subject).

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes. The decrease in expression can be complete or partial. In some embodiments, the knockdown is at least, or about, 10%, 20%, 30%, 40%, 50%, 60%, 7%, 80%, 90%, 95%, or 99% as compared to a gene that has not been knockdowned in the same cell, organism or system. The knockdown can be effectuated by any technique, such as gene editing, siRNA, antisense, or other gene modification methods.

The term “knockout” as used herein refers to the ablation of gene expression of one or more genes. For example, a “knockout” can mean that the entire gene has been removed or a portion of the gene has been removed or mutated in a manner that leads to the complete inhibition of the product encoded by the gene. Therefore, a knockout can be a complete removal of the gene or an exon of the gene. A knockout can also be accomplished by introducing a mutation in the gene sequence that prevents the expression of the gene product (e.g. protein) from the gene.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell. Molecules may be modified in many ways, including chemically, structurally, and functionally. Cells may be modified through the introduction of nucleic acids. In reference to a protein or gene, a modified protein or gene can refer to a protein or gene having a mutation, such as a insertion, deletion, point mutation, or any combination thereof.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “oligonucleotide” typically refers to polynucleotides. It will be understood that when a nucleotide sequence is represented by a DNA sequence (i.e., A, T, C, G), this also includes an RNA sequence (i.e., A, U, C, G) in which “U” replaces “T.” A guide RNA (gRNA) is a non-limiting example of an “oligonucleotide.”

In the context of the present embodiments, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition or other composition provided for herein (e.g., viral vector), includes, e.g., subcutaneous (s.c.), intravenous (i.v.), intramuscular (i.m.), or intrasternal injection, or infusion techniques.

The term “polynucleotide” or “nucleic acid molecule” as used herein is defined as a chain of nucleotides. Furthermore, nucleic acid molecules are polymers of nucleotides. Thus, nucleic acid molecules and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “targeting moiety” refers to a molecule, such as a protein, that can be used to target a cell or virus to a bind or interact with a cell expressing the target. In some embodiments, the targeting moiety is an antibody or other type of targeting moiety (e.g. a scFv, an antigen binding domain, a DARPIN, a VHH, or a FN3 domain). In some embodiments, the targeting moiety is express on the surface of a vector to target the vector to a cell expressing the binding partner (e.g., antigen) of the targeting moiety.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (P) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. For example, in some embodiments, the cell is transfected, transduced by a vector comprising the nucleic acid molecule. In some embodiments, the cell is transfected with a plasmid comprising the nucleic acid molecule.

To “treat” a disease as the term is used herein, means to reduce the frequency or severity of at least one sign or symptom of a disease or disorder experienced by a subject. As used in reference to a solid tumor, the term “treat” can be mean the reduction in the size of the solid tumor or in the number of locations a tumor is found either at its origin or that has metastasized.

A “vector” is a composition of matter which comprises a nucleic acid and which can be used to deliver the nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, lipid nanoparticles, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects can be presented in a range format. It should be understood that the description in range format is merely for convenience and brevity and should not be construed as an inflexible limitation on the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Immune Cells

The present disclosure provides modified immune cells or precursors thereof (e.g., T cells) for use in immunotherapy (e.g. CAR T cells). In certain aspects, the disclosure provides a modified immune cell or precursor cell thereof (e.g., T cell) comprising a chimeric antigen receptor (CAR), wherein the endogenous CD5 gene has been disrupted and/or knocked-out of the cell.

For example, in some embodiments, the endogenous CD5 gene can be mutated to inhibit or reduce the expression of the CD5 gene. As provided for herein, the CD5 gene can be mutated to include an insertion, substitution, deletion, or any combination thereof, to inhibit the function or expression of the CD5 gene product, protein. In some embodiments, the mutation is an exon of the CD5 gene. The CD5 gene is believed to have at least 11 exons, which are described in Padilla et al., Immunogenetics, 2000 October; 51(12):993-1001, which is hereby incorporated by reference in its entirety. In some embodiments, the mutation is in exon 1 of the CD5 gene. In some embodiments, the mutation is in exon 2 of the CD5 gene. In some embodiments, the mutation is in exon 3 of the CD5 gene. In some embodiments, the mutation is in exon 4 of the CD5 gene. In some embodiments, the mutation is in exon 5 of the CD5 gene. In some embodiments, the mutation is in exon 6 of the CD5 gene. In some embodiments, the mutation is in exon 7 of the CD5 gene. In some embodiments, the mutation is in exon 8 of the CD5 gene. In some embodiments, the mutation is in exon 9 of the CD5 gene. In some embodiments, the mutation is in exon 10 of the CD5 gene. In some embodiments, the mutation is in exon 11 of the CD5 gene.

The modified cell can comprise any CAR known in the art as well as those described in detail elsewhere herein. A CAR comprises affinity for an antigen on a target cell. Accordingly, such modified cells possess the specificity directed by the CAR that is expressed therein. In some embodiments, the modified cell comprises a CAR that comprises an antigen binding domain that binds to CD5, which can be referred to as a “CD5 CAR.” Without being bound to any particular theory, a cell expressing the CD5 CAR would possess specificity for CD5 on a target cell. In certain embodiments, the disclosure provides a modified immune cell or precursor cell thereof (e.g., T cell) comprising a CD5 CAR, wherein the endogenous CD5 gene has been disrupted, mutated, knocked-down and/or knocked-out as provided for herein By including a CD5 CAR in a cell where the CD5 gene is mutated, the CAR should avoid targeting other CAR-T cells expressing the CD5 CAR. This can then be used to avoid “fratricide” that can occur if the endogenous CD5 protein in the T cell were expressed along with the CAR. In some embodiments, the CAR binds to a solid tumor antigen, such as, but not limited to, those provided for herein.

The present disclosure provides gene edited modified cells. In some embodiments, a modified cell (e.g., a modified cell comprising a CAR) of the present disclosure is genetically edited to disrupt the expression of an endogenous gene locus encoding CD5. In some embodiments, the gene-edited immune cells (e.g., T cells) have a downregulation, reduction, deletion, elimination, knockdown, knockout or disruption in expression of the endogenous CD5. In some embodiments, the cell comprises a gene edit in exon 1 of the CD5 gene. In some embodiments, the cell comprises a gene edit in exon 2 of the CD5 gene In some embodiments, the cell comprises a gene edit in exon 3 of the CD5 gene In some embodiments, the cell comprises a gene edit in exon 4 of the CD5 gene. In some embodiments, the cell comprises a gene edit in exon 5 of the CD5 gene. In some embodiments, the cell comprises a gene edit in exon 6 of the CD5 gene. In some embodiments, the cell comprises a gene edit in exon 7 of the CD5 gene. In some embodiments, the cell comprises a gene edit in exon 8 of the CD5 gene. In some embodiments, the cell comprises a gene edit in exon 9 of the CD5 gene. In some embodiments, the cell comprises a gene edit in exon 10 of the CD5 gene. In some embodiments, the cell comprises a gene edit in exon 11 of the CD5 gene.

Various gene editing technologies are known to those skilled in the art. Gene editing technologies include, without limitation, homing endonucleases, zinc-finger nucleases (ZFNs), transcription activator-like effector (TALE) nucleases (TALENs), and clustered regularly interspaced short palindromic repeats (CRISPR) (e.g. CRISPR/Cas9). Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM). Accordingly, one of skill in the art can utilize, or would be able to select, the appropriate gene editing technology to produce the modified cell..

In certain embodiments, the present embodiments provides a modified immune cell or precursor cell thereof (e.g., a T cell) comprising a CRISPR-mediated modification in an endogenous gene locus encoding CD5 that is capable of downregulating gene expression of endogenous CD5, and an exogenous CAR as described herein. The gene locus can comprise one or more of the exons described herein for CD5.

In certain embodiments, the CRISPR-mediated modification is introduced via a CRISPR/Cas9 system, comprising a Cas9 enzyme and at least one guide RNA (gRNA.) In certain embodiments, the guide RNA comprises a guide sequence that is sufficiently complementary with a target sequence in the endogenous gene locus CD5. In certain embodiments, the guide RNA comprises a guide sequence that is sufficiently complementary with a target sequence in exon 2 of the endogenous CD5 gene locus. In certain embodiments, the guide RNA comprises or consists of the nucleic acid sequence set forth in SEQ ID NO: 45.

Non-limiting types of CRISPR-mediated modifications include a substitution, an insertion, a deletion, and an insertion/deletion (INDEL). The modification can be located in any part of the endogenous gene locus encoding CD5, including but not limited to an exon, a splice donor, or a splice acceptor. In certain embodiments, the modification is in exon 2 of CD5.

In some aspects, the disruption is carried out by gene editing using an RNA-guided nuclease such as a CRISPR-Cas system, such as CRISPR-Cas9 system, specific for the gene (e.g., CD5) being disrupted. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and TCR T cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

The CRISPR example is a non-limiting example of gene editing technologies or methods that can be used. Other nucleases and gene editing systems can also be used, including those, but not limited to, those that are provided in WO2022232638, WO2022232638, WO2022198080, WO2022159758, WO2022159742, WO2022066335, WO2022056324, WO2022056301, WO2022046662, WO2021178933, WO2021226363, WO2021226369, WO2021202568, WO2021202559, WO2021178934, WO2020168234, each of which is hereby incorporated by reference in its entirety.

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas system is derived from a Cas9 nuclease. Exemplary Cas9 nucleases that may be used include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that can be used. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

In some embodiments, a gene edited modified cell of the present disclosure is edited using CRISPR/Cas9 or other nuclease, such as those provided for herein, to disrupt an endogenous gene locus encoding CD5. Suitable gRNAs for use in disrupting CD5 are known in the art and, for example, are set forth in SEQ ID NO: 28. It will be understood to those of skill in the art that guide RNA sequences may be recited with a thymidine (T) or a uridine (U) nucleotide.

Accordingly, in some embodiments a modified immune cell or precursor cell thereof is provided comprising a nuclease-mediated modification in an endogenous gene locus encoding CD5, wherein the modification is capable of downregulating gene expression of endogenous CD5; and a CAR comprising affinity for an antigen on a target cell. The gene modification, however, can be made using other gene editing technologies and the reference to a CRISPR-mediated modification as provided for herein is simply for illustrative purposes only and other types of gene-editing mediated modifications can be substituted.

In some embodiments, the nucleic acid encoding a CAR is inserted into an exon (e.g. exon 1, exon 2, exon 3, exon 4, exon 5, exon 6, exon 7, exon 8, exon 9, exon 10, or exon 11) of the endogenous gene locus encoding CD5. In some embodiments, the nucleic acid encoding a CAR is inserted into a splice donor of the endogenous gene locus encoding CD5. In some embodiments, the nucleic acid encoding a CAR is inserted into a splice acceptor of the endogenous gene locus encoding CD5. In some embodiments, the insertion point is exon 2.

In certain embodiments, the modified cell is an autologous cell in reference to the subject that the cell is eventually administered to. In certain embodiments, the modified cell is a cell isolated from a human subject. In certain embodiments, the modified cell is a modified immune cell. In certain embodiments, the modified cell is a modified T cell. The modified T cell as provided for herein can comprise the CD5 modification/mutation and the CAR.

In some aspects, the provided compositions and methods include those in which at least or greater than about 50%, 60%, 65%, 70%, 75%, 80%, 85%, 90% or 95% of immune cells in a composition of immune cells contain the desired genetic modification. For example, about 50%, 60%, 65%, 70%, 75%, 80%, 85%, 90% or 95% of immune cells in a composition of cells into which an agent (e.g. gRNA/Cas9 or other gRNA and gene editing nuclease) for knockout or genetic disruption of endogenous CD5 was introduced, contain the genetic disruption, do not express the targeted endogenous polypeptide, and/or do not contain a contiguous and/or functional copy of the targeted gene. In some embodiments, the methods, compositions and cells according to the present disclosure include those in which at least or greater than about 50%, 60%, 65%, 70%, 75%, 80%, 85%, 90% or 95% of cells in a composition of cells into which an agent (e.g. gRNA/Cas9 or other gRNA and gene editing nuclease) for knockout or genetic disruption of a targeted gene was introduced do not express the targeted polypeptide, such as on the surface of the immune cells. In some embodiments, at least or greater than about 50%, 60%, 65%, 70%, 75%, 80%, 85%, 90% or 95% of cells in a composition of cells into which an agent (e.g. gRNA/Cas9 or other gRNA and gene editing nuclease) for knockout or genetic disruption of the targeted gene was introduced are knocked out in both alleles, i.e. comprise a biallelic deletion, in such percentage of cells.

In some embodiments, provided are compositions and methods in which the Cas9-mediated cleavage efficiency (% indel) in or near the targeted gene (e.g. within or about within 100 base pairs, within or about within 50 base pairs, or within or about within 25 base pairs or within or about within 10 base pairs upstream or downstream of the cut site) is at least or greater than about 50%, 60%, 65%, 70%, 75%, 80%, 85%, 90% or 95% in cells of a composition of cells into which an agent (e.g. gRNA/Cas9 or other gRNA and gene editing nuclease) for knockout or genetic disruption of a targeted gene has been introduced.

In some embodiments, the provided cells, compositions and methods results in a reduction or disruption of signals delivered via the endogenous in at least or greater than about 50%, 60%, 65%, 70%, 75%, 80%, 85%, 90% or 95% of cells in a composition of cells into which an agent (e.g. gRNA/Cas9 or other gRNA and gene editing nuclease) for knockout or genetic disruption of a targeted gene was introduced.

In certain embodiments, the modified cell of the present disclosure is genetically edited to disrupt the expression of CD5. As described elsewhere herein, disruption of CD5 is shown by the present disclosure to enhance immune cell (e.g., CAR T cell) function.

In some embodiments, the modified cell of the present disclosure is genetically edited to disrupt the expression of an additional endogenous gene. For example, the cell may be further edited to disrupt an endogenous PDCD1 gene product (e.g. Programmed Death 1 receptor; PD-1). Disrupting the expression of endogenous PD-1 may create “checkpoint” resistant modified cells, resulting in increased tumor control. Checkpoint resistant modified cells may also be created by disrupting the expression of, for example, without limitation, the Adenosine A2A receptor (A2AR), B7-H3 (CD276), B7-H4 (VTCN1), the B and T Lymphocyte Attenuator protein (BTLA/CD272), CD96, the Cytotoxic T-Lymphocyte Associated protein 4 (CTLA-4/CD152), Indoleamine 2,3-dioxygenase (IDO), the Killer-cell Immunoglobulin-like Receptor (KIR), the Lymphocyte Activation Gene-3 (LAG3), the T cell immunoreceptor with Ig and ITIM domains (TIGIT), T-cell Immunoglobulin domain and Mucin domain 3 (TIM-3), the CD200 Receptor (CD200R), or the V-domain Ig suppressor of T cell activation (VISTA). The cell may be further edited to disrupt other endogenous genes, including but not limited to, the T Cell Receptor Alpha Chain (TRAC), T Cell Receptor Beta Chain (TRBC), Programmed Death-1 (PD-1) Ligand 1 (PDL1), and Transforming Growth Gactor β (TGF-β) Receptor (TGFβR).

In some embodiments, compositions according to the provided disclosure that comprise cells engineered with a CAR and comprise the reduction, deletion, elimination, knockout or disruption in expression of an endogenous CD5 gene retain the functional property or activities of the receptor compared to the receptor expressed in engineered cells of a corresponding or reference composition comprising the receptor but do not comprise the genetic disruption of a gene or express the polypeptide when assessed under the same conditions. In some embodiments, the engineered cells of the provided compositions retain a functional property or activity compared to a corresponding or reference composition comprising engineered cells in which such are engineered with the CAR but do not comprise the genetic disruption or express the targeted polypeptide when assessed under the same conditions. In some embodiments, the cells retain cytotoxicity, proliferation, survival or cytokine secretion compared to such a corresponding or reference composition.

In some embodiments, the immune cells in the composition retain a phenotype of the immune cell or cells compared to the phenotype of cells in a corresponding or reference composition when assessed under the same conditions. In some embodiments, cells in the composition include naive cells, effector memory cells, central memory cells, stem central memory cells, effector memory cells, and long-lived effector memory cells. In some embodiments, the percentage of T cells, or T cells expressing the CAR, and comprising the genetic disruption of a CD5 exhibit a non-activated, long-lived memory or central memory phenotype that is the same or substantially the same as a corresponding or reference population or composition of cells engineered with the recombinant receptor but not containing the genetic disruption. In some embodiments, such property, activity or phenotype can be measured in an in vitro assay, such as by incubation of the cells in the presence of an antigen targeted by the CAR, a cell expressing the antigen and/or an antigen-receptor activating substance. In some embodiments, any of the assessed activities, properties or phenotypes can be assessed at various days following electroporation or other introduction of the agent, such as after or up to 3, 4, 5, 6, 7 days. In some embodiments, such activity, property or phenotype is retained by at least 80%, 85%, 90%, 95% or 100% of the cells in the composition compared to the activity of a corresponding composition containing cells engineered with the recombinant receptor but not comprising the genetic disruption of the targeted gene when assessed under the same conditions.

As used herein, reference to a “corresponding composition” or a “corresponding population of immune cells” (also called a “reference composition” or a “reference population of cells”) refers to immune cells (e.g., T cells) obtained, isolated, generated, produced and/or incubated under the same or substantially the same conditions, except that the immune cells or population of immune cells were not introduced with the agent. In some aspects, except for not containing introduction of the agent, such immune cells are treated identically or substantially identically as immune cells that have been introduced with the agent, such that any one or more conditions that can influence the activity or properties of the cell, including the upregulation or expression of the inhibitory molecule, is not varied or not substantially varied between the cells other than the introduction of the agent.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, CAR-expressing cells can be detected by flow cytometry or other immunoaffinity based method for expression of a marker unique to such cells, and then such cells can be co-stained for another T cell surface marker or markers.

In some embodiments, the cells, compositions and methods provide for the deletion, knockout, disruption, or reduction in expression of the target gene in immune cells (e.g. T cells) to be adoptively transferred (such as cells engineered to express a CAR). In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) one or more nucleic acid encoding a CAR and an agent or agents that is capable of disrupting, a gene that encode the endogenous receptor to be targeted. As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the nucleic acid encoding a genetically engineered antigen receptor and the step of introducing the agent (e.g. Cas9/gRNA RNP) can occur simultaneously or sequentially in any order. In some embodiments, subsequent to introduction of the exogenous receptor and one or more gene editing agents (e.g. Cas9/gRNA RNP), the cells are cultured or incubated under conditions to stimulate expansion and/or proliferation of cells.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time. In some embodiments, the genetically engineered cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells were not introduced with an agent that reduces expression of or disrupts a gene encoding an endogenous receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In certain embodiments, the immune cell or precursor cell thereof is a T cell. In certain embodiments, the T cell is a human T cell. In certain embodiments, the cell is an autologous cell (e.g. an autologous T cell).

The modified cells can comprise any chimeric antigen receptor (CAR) disclosed herein.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time.

In some embodiments, the degree or extent of persistence of administered cells can be detected or quantified after administration to a subject. For example, in some aspects, quantitative PCR (qPCR) is used to assess the quantity of cells expressing the CAR in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the modified cell can be used to distinguish the administered cells from endogenous cells in a subject.

Disruption, mutation, or reduction of CD5 can be carried out by any method known in the art including but not limited to, gene editing, miRNA, siRNA, a drug, an antibody, an inhibitor, and the like.

The cells provided for herein can also be generated in vivo by administering a vector, such as a virus to transduce the T cell in vivo. The T cell can be targeted by modifying the vector with a targeting moiety. Examples of transduction systems and vectors that can be used to produce the cells in vivo are described in, but not limited to, U.S. Publication Application No. 20210353543, U.S. Pat. Nos. 10,064,958, 9,486,539, PCT Publication No. WO/2021/202604, U.S. Publication No. 20210228627, U.S. Publication No. 20210198698, and U.S. Publication No. 20210283179, each of which is hereby incorporated by reference in its entirety.

### C. Methods of Treatment

The modified cells described herein (e.g., CAR T cells wherein endogenous CD5 has been disrupted, mutated, and/or knocked out) may be included in a composition for immunotherapy for treating solid tumors. The composition may include a pharmaceutical composition and further include a pharmaceutically acceptable carrier. A therapeutically effective amount of the pharmaceutical composition comprising the modified cells may be administered.

In some embodiments, the disclosure includes a method of treating a solid tumor in a subject in need thereof, comprising administering to the subject a population of modified immune cells or precursor cells thereof (e.g. CAR T cells wherein endogenous CD5 has been disrupted, mutated, and/or knocked out) disclosed herein such that the expression of the CD5 is inhibited, reduced or eliminated. In another aspect, the embodiments are provided that include a method for adoptive cell transfer therapy comprising administering to a subject in need thereof a modified immune cell or precursor cell thereof as provided herein (e.g. CAR T cells wherein endogenous CD5 has been disrupted and/or knocked out).

In some embodiments, methods of treating a solid tumor in a subject in need thereof are provided, wherein the methods comprise administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen.

In some embodiments, methods of treating a solid tumor in a subject in need thereof are provided, wherein the methods comprise administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, wherein the growth of the tumor is inhibited for at least 10, 20, 30, 40, 50, 60, 70, 80, 90, 100, 120, 150, 200, 240 days.

In some embodiments, methods of treating a solid tumor with enhanced efficacy in a subject in need thereof are provided, wherein the methods comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, wherein the efficacy of the population of immune cells comprising the mutated endogenous CD5 gene is greater than a population of immune cells comprising a non-mutated endogenous CD5 gene. In some embodiments, the enhanced efficacy is an increase in cytotoxicity against the solid tumor cells. In some embodiments, cytotoxicity against the solid tumor cells is increased at least 10, 20, 30, 40, 50, 60, 70, 80, 90, 100, 200, or 300% as compared to a population of immune cells without a mutated CD5 gene.

In some embodiments, at least 75%, 80%, 85%, 90%, 91%, 92%, 93%, 94%, 95%, 96%, 97%, 98%, 99%, or 100% the population of immune cells comprise the mutated endogenous CD5 gene. In some embodiments, at least 50%, 55%, 60%, 65%, 75%, 80%, 85%, 90%, 91%, 92%, 93%, 94%, 95%, 96%, 97%, 98%, 99%, or 100% the population of immune cells comprise the heterologous chimeric antigen receptor (CAR).

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells can be administered to an animal, preferably a mammal, even more preferably a human, to treat a cancer, such as a solid tumor. In addition, the cells of can be used for the treatment of any condition related to a cancer, especially a cell-mediated immune response against a tumor cell(s), where it is desirable to treat or alleviate the disease. The types of cancers to be treated with the modified cells or pharmaceutical compositions provided herein include, but are not limited to, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. Adult tumors/cancers and pediatric tumors/cancers are also included. In some embodiments, the cancer is a carcinoma. In one embodiment, the cancer is a sarcoma. In some embodiments, the cancer is a leukemia. In some embodiments, the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells provided for herein are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells provided for herein are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells to be administered may be autologous, with respect to the subject undergoing therapy. In some embodiments, the cells are allogeneic with respect to the subject undergoing therapy.

The administration of the cells may be carried out in any convenient manner known to those of skill in the art. The cells may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at or about 1×10⁵ CD4+ and/or CD8+ cells/kg, 1.5×10⁵ CD4+ and/or CD8+ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 108 and 10¹² or between about 10¹⁰ and 10¹¹ CD8+ cells. In some embodiments, about 1×10⁶ to about 1×10⁹, about 1×10⁷ to about 1×10⁹, about 1×10⁸ to about 1×10⁹, about 2×10⁸ to about 1×10⁹, about 3×10⁸ to about 1×10⁹, about 3×10⁸ to about 1×10⁹, about 3×10⁸ to about 1×10⁹, about 4×10⁸ to about 1×10⁹, about 5×10⁸ to about 1×10⁹, about 6×10⁸, about 7×10⁸ to about 1×10⁹, about 8×10⁸, about 9×10⁸ to about 1×10⁹ of the immune cells are administered to the subject being treated.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine. In some embodiments, a specific dosage regimen of the present disclosure does not include a lymphodepletion step prior to the administration of the modified T cells.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

The modified cells can be administered in dosages and routes and at times to be determined in appropriate pre-clinical and clinical experimentation and trials. Cell compositions may be administered multiple times at dosages within these ranges. Administration of the cells may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the embodiments provide for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

As such, the modified immune cells provided for herein when used in a method of treatment as described herein, enhances the ability of the modified immune cells in carrying out their function. Accordingly, the embodiments provided for herein provide a method for enhancing a function of a modified immune cell for use in a method of treatment as described herein.

In one aspect, the embodiments include a method of treating cancer in a subject in need thereof, comprising administering to the subject any one of the modified immune or precursor cells provided for herein. Yet another aspect of the embodiments include a method of treating cancer in a subject in need thereof, comprising administering to the subject a modified immune or precursor cell generated by any one of the methods disclosed herein.

In certain embodiments, the method comprises administering to the subject a modified immune cell comprising a chimeric antigen receptor (CAR), wherein the CAR comprises an antigen binding domain, a transmembrane domain, and an intracellular domain, and wherein the endogenous CD5 gene has been mutated, disrupted, or otherwise knocked-out of the cell as provided for herein.

In the various embodiments of the methods disclosed herein, the subject can be administered any CAR known in the art or disclosed herein. The CAR can be specific for any tumor associated antigen (TAA) or tumor specific antigen (TSA) known to one of ordinary skill in the art.

In certain embodiments, the CAR is a CD5 CAR. In certain embodiments, the CAR comprises a complementarity determining region (CDR) comprising the amino acid sequence selected from the group consisting of SEQ ID NOs: 27, 28, 29, 30, 31, 32, 39, 40, 41, 42, 43, or 44. In certain embodiments, the CAR comprises an antigen binding domain comprising a heavy chain variable region comprising an amino acid sequence selected from the group consisting of SEQ ID NOs: 19, 25, and 37 and/or a light chain variable region comprising an amino acid sequence selected from the group consisting of SEQ ID NOs: 20, 26, and 38. In certain embodiments, the CAR comprises an scFv comprising an amino acid sequence selected from the group consisting of SEQ ID NOs: 17, 18, 23, 24, 35, or 36.

In certain embodiments, the subject is administered a CAR, wherein the CAR comprises a nucleic acid sequence encoded by any one of SEQ ID NOs: 1-6. In certain embodiments, the CAR comprises an amino acid sequence selected from the group consisting of SEQ ID NOs: 15, 16, 21, 22, 33, or 34.

### C. Chimeric Antigen Receptors

The present disclosure provides compositions and methods for modified immune cells or precursors thereof, e.g., modified T cells, comprising a chimeric antigen receptor (CAR) wherein endogenous CD5 has been mutated, modified, disrupted, or knocked out. Thus, in some embodiments, the immune cell has been genetically modified to express the CAR. CARs can comprise an antigen binding domain, a transmembrane domain, and an intracellular domain.

The antigen binding domain of the CAR may be operably linked to another domain of the CAR, such as the transmembrane domain or the intracellular domain for expression in the cell. In some embodiments, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein or known, any of the intracellular domains or cytoplasmic domains described herein or known, or any of the other domains described herein that may be included in a CAR. A CAR may also include a hinge domain. A CAR may also include a spacer domain. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell, such as, but not limited to, a solid tumor cell.

In some embodiments embodiment, the target cell antigen is a solid tumor antigen, such as, but not limited to mesothelin. In some embodiments, it is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-1, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS. In some embodiments, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, MY-ESO-1 TCR, MAGE A3 TCR, and the like.

Depending on the desired antigen to be targeted, the CAR can be engineered to include the appropriate antigen binding domain that is specific to the desired antigen target. For example, if CD5 is the desired antigen that is to be targeted, an antibody for CD5 can be used as the antigen bind moiety for incorporation into the CAR.

In some embodiments, the target cell antigen is CD5. As such, in some embodiments, a CAR has affinity for CD5 on a target cell. This should not be construed as limiting in any way, as a CAR having affinity for any target antigen is suitable for use in a composition or method.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In some embodiments, a CAR having affinity for CD5 on a target cell may comprise a CD5 binding domain.

In some embodiments, a CAR may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). In some embodiments, the target antigen binding domain is selected from the group consisting of a target antigen-specific antibody, a target antigen-specific Fab, and a target antigen-specific scFv. In some embodiments, a target antigen binding domain is a target antigen-specific antibody. In some embodiment, a target antigen binding domain is a target antigen-specific Fab. In some embodiments, a target antigen binding domain is a target antigen-specific scFv.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., PSCA binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration.

The linker can be rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ(SEQ ID NO:46), (GGGS)ₙ(SEQ ID NO:47), and (GGGGS)ₙ (SEQ ID NO:48), where n represents an integer of at least 1. Exemplary linker sequences can comprise amino acid sequences including, without limitation, GGSG (SEQ ID NO:49), GGSGG (SEQ ID NO:50), GSGSG (SEQ ID NO:51), GSGGG (SEQ ID NO:52), GGGSG (SEQ ID NO:53), GSSSG (SEQ ID NO:54), GGGGS (SEQ ID NO:55), GGGGSGGGGSGGGGS (SEQ ID NO:56) and the like. Those of skill in the art would be able to select the appropriate linker sequence. In some embodiments, an antigen binding domain comprises a heavy chain variable region (VH) and a light chain variable region (VL), wherein the VH and VL are separated by a linker sequence having the amino acid sequence GGGGSGGGGSGGGGS (SEQ ID NO:56), which may be encoded by the nucleic acid sequence GGTGGCGGTGGCTCGGGCGGTGGTGGGTCGGGTGGCGGCGGATCT (SEQ ID NO:57).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof.

In certain embodiments, the antigen binding domain of the CAR targets a solid tumor antigen. In some embodiments, the antigen binding domain in the CAR is an anti-solid-tumor antigen scFV. In some embodiments, the antigen binding domain is an anti-solid tumor antigen antibody.

In certain embodiments, the antigen binding domain comprises a heavy chain variable region that comprises three heavy chain complementarity determining regions (HCDRs) and a light chain variable region that comprises three light chain complementarity determining regions (LCDRs).

In certain embodiments, the modified cell comprises a CAR comprising an antigen binding domain capable of binding CD5, wherein the antigen binding domain comprises a complementarity determining region (CDR) comprising the amino acid sequence of any one of SEQ ID NOs: 27, 28, 29, 30, 31, 32, 39, 40, 41, 42, 43, or 44.

In certain embodiments, the CAR comprises an antigen binding domain capable of binding CD5, wherein HCDR1 comprises the amino acid sequence of SEQ ID NO: 27, HCDR2 comprises the amino acid sequence of SEQ ID NO: 28, HCDR3 comprises the amino acid sequence of SEQ ID NO: 29, LCDR1 comprises the amino acid sequence of SEQ ID NO: 30, LCDR2 comprises the amino acid sequence of SEQ ID NO: 31, and LCDR3 comprises the amino acid sequence of SEQ ID NO: 32.

In certain embodiments, the CAR comprises an antigen binding domain capable of binding CD5, wherein HCDR1 comprises the amino acid sequence of SEQ ID NO: 39, HCDR2 comprises the amino acid sequence of SEQ ID NO: 40, HCDR3 comprises the amino acid sequence of SEQ ID NO: 41, LCDR1 comprises the amino acid sequence of SEQ ID NO: 42, LCDR2 comprises the amino acid sequence of SEQ ID NO: 43, and LCDR3 comprises the amino acid sequence of SEQ ID NO: 44.

In certain embodiments, the CAR comprises an antigen binding domain capable of binding CD5, wherein the antigen binding domain comprises a heavy chain variable region comprising the amino acid sequence of SEQ ID NO: 19 and/or a light chain variable region comprising the amino acid sequence of SEQ ID NO: 20. In certain embodiments, the heavy chain variable region comprises the amino acid sequence of SEQ ID NO: 25 and/or a light chain variable region comprises the amino acid sequence of SEQ ID NO: 26. In certain embodiments, the heavy chain variable region comprises the amino acid sequence of SEQ ID NO: 37 and/or a light chain variable region comprises the amino acid sequence of SEQ ID NO: 38.

In certain embodiments, the CAR comprises an antigen binding domain capable of binding CD5, wherein the antigen binding domain is a scFv comprising the amino acid sequence set forth in any one of SEQ ID NOs: 17, 18, 23, 24, 35, or 36.

Tolerable variations of the antigen binding domain sequences will be known to those of skill in the art. For example, in some embodiments the antigen binding domain comprises an amino acid sequence that has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 17-20, 23-32, or 35-44.

### Transmembrane Domain

CARs of may comprise a transmembrane domain that connects the antigen binding domain of the CAR to the intracellular domain of the CAR. The transmembrane domain of a CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A CAR of may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a CAR includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ(SEQ ID NO:46) and (GGGS)ₙ(SEQ ID NO:47), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:49), GGSGG (SEQ ID NO:50), GSGSG (SEQ ID NO:51), GSGGG (SEQ ID NO:52), GGGSG (SEQ ID NO:53), GSSSG (SEQ ID NO:54), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:58); CPPC (SEQ ID NO:59); CPEPKSCDTPPPCPR (SEQ ID NO:60) (see, e.g., Glaser et al., J. Biol. Chem. (2005) 280:41494-41503); ELKTPLGDTTHT (SEQ ID NO:61); KSCDKTHTCP (SEQ ID NO:62); KCCVDCP (SEQ ID NO:63); KYGPPCP (SEQ ID NO:64); EPKSCDKTHTCPPCP (SEQ ID NO:65) (human IgG1 hinge); ERKCCVECPPCP (SEQ ID NO:66) (human IgG2 hinge); ELKTPLGDTTHTCPRCP (SEQ ID NO:67) (human IgG3 hinge); SPNMVPHAHHAQ (SEQ ID NO:68) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:69); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A CAR also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the (chain of the T cell receptor complex or any of its homologs, e.g., 1 chain, FcsRIT and R chains, MB 1 (Iga) chain, B29 (Ig) chain, etc., human CD3 zeta chain, CD3 polypeptides (A, 6 and P), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In some embodiments, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a CAR include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a CAR include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in a CAR of the present disclosure includes a DAP10/CD28 type signaling chain. In one embodiment, an intracellular signaling domain suitable for use in a CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

In some embodiments, the anti-CD5 CAR comprises the amino acid sequence set forth in any one of SEQ ID NOs: 15, 16, 21, 22, 33, or 34. In one embodiment, the anti-CD5 CAR is encoded by a nucleic acid sequence selected from the group consisting of SEQ ID NOs: 1-6.

Tolerable variations of the CAR sequences will be known to those of skill in the art. For example, in some embodiments the CAR comprises an amino acid sequence that has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NO: 15, 16, 21, 22, 33, or 34. In some embodiments the CAR is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least or 99% sequence identity to the nucleic acid sequence set forth in SEQ ID NO: 1-6.

The embodiments can include any one of: a CAR, a nucleic acid encoding a CAR, a vector comprising a nucleic acid encoding a CAR, a cell comprising a CAR, a cell comprising a nucleic acid encoding a CAR, and a cell comprising a vector comprising a nucleic acid encoding a CAR.

CD5-17L2H-3045 CAR
(SEQ ID NO: 1)
ggatccAACATTGTACTGACGCAAAGCCCCTCATCTTTGTCTGAGTCACTCGGCGGC
AAAGTAACCATCACATGCAAGGCCAGTCAAGACATCAATAAATATATTGCTTGG
TATCAGTATAAACCCGGCAAGGGGCCGCGACTGCTGATTCACTACACGAGTACC
TTGCAACCGGGCATTCCGAGCCGATTTAGTGGCAGTGGCTCAGGTCGCGATTACT
CATTCTCAATAAGTAATCTCGAACCGGAAGACATAGCTACTTATTATTGCTTGCA
GTACGATAATTTGTGGACCTTCGGGGGTGGTACAAAGTTGGAAATAAAGggtggcgg
agggagcggcggtggaggaagcggaggcggaggttccGAGGTCCAACTCGTAGAATCAGGTCCCGG
ATTGGTGCAACCATCCCAGAGCCTCTCTATTACATGCACGGTCTCTGGATTTAGT
CTGACCAATTACGATGTGCATTGGGTGCGCCAGTCTCCCGGCAAGGGGTTGGAA
TGGCTTGGCGTTATATGGAACTACGGAAATACAGACTATAACGCCGCGTTTATCT
CTCGGCTGAGTATACGGAAAGACAGTAGTAAATCCCAGGTCTTTTTTACGATGTC
ATCCCTGCAAACGCCAGATACCGCAATATATTACTGCGCCAGGAACCACGGTGA
TGGTTATTATAATTGGTACTTCGATGTGTGGGGTACTGGCACTACAGTCACAGTA
TCTTCAtctaga
CD5-9H2L-3048 CAR
(SEQ ID NO: 2)
ggatcc CAG GTC CAG CTG AAA GAA AGC GGT CCA GAG CTG GAA AAA CCC
GGT GCG AGC GTC AAA ATA TCA TGT AAA GCA AGC GGG TAT TCA TTC ACC
GCG TAC TCT ATG AAC TGG GTT AAG CAA AAC AAC GGT ATG TCC TTG GAG
TGG ATA GGG TCT ATC GAC CCG TAT TAT GGG GAC ACA AAA TAC GCG CAG
AAA TTC AAG GGG AAG GCC ACC CTG ACC GTA GAT AAA GCT AGT TCT ACT
GCG TAC TTG CAA CTG AAA AGC CTC ACT TCT GAG GAC TCT GCC GTC TAC
TAC TGT GCT CGG CGA ATG ATA ACG ACG GGG GAC TGG TAT TTC GAT GTT
TGG GGT ACA GGG ACT ACG GTG ACT GTC AGT
AGCggtggcggagggagcggcggtggaggaagcggaggcggaggttcc CAT ATC GTC TTG ACT CAA
TCA CCT AGT TCT TTG TCT GCG TCC CTT GGC GAC CGA GTC ACC ATA TCT
TGC AGA GCG TCA CAG GAC ATT TCA ACG TAC CTC AAC TGG TAT CAG CAA
AAA CCG GAC GGG ACT GTC AAG CTC TTG ATC TTC TAC ACT TCC AGA CTC
CAC GCC GGG GTG CCA AGC AGA TTT AGT GGC TCT GGC AGC GGG ACA CAC
CAT AGT CTT ACA ATC AGC AAT CTT GAG CAA GAA GAC ATA GCC ACG TAT
TTC TGC CAG CAA GGT AAC TCA CTT CCG TTC ACG TTT GGT AGT GGC ACC
AAA CTG GAG ATA AAA tccgga
CD5-9L2H-3049 CAR
(SEQ ID NO: 3)
Ggatcc CAT ATC GTC TTG ACT CAA TCA CCT AGT TCT TTG TCT GCG TCC CTT
GGC GAC CGA GTC ACC ATA TCT TGC AGA GCG TCA CAG GAC ATT TCA ACG
TAC CTC AAC TGG TAT CAG CAA AAA CCG GAC GGG ACT GTC AAG CTC TTG
ATC TTC TAC ACT TCC AGA CTC CAC GCC GGG GTG CCA AGC AGA TTT AGT
GGC TCT GGC AGC GGG ACA CAC CAT AGT CTT ACA ATC AGC AAT CTT GAG
CAA GAA GAC ATA GCC ACG TAT TTC TGC CAG CAA GGT AAC TCA CTT CCG
TTC ACG TTT GGT AGT GGC ACC AAA CTG GAG ATA AAA
ggtggcggagggagcggcggtggaggaagcggaggcggaggttcc CAG GTC CAG CTG AAA GAA AGC
GGT CCA GAG CTG GAA AAA CCC GGT GCG AGC GTC AAA ATA TCA TGT AAA
GCA AGC GGG TAT TCA TTC ACC GCG TAC TCT ATG AAC TGG GTT AAG CAA
AAC AAC GGT ATG TCC TTG GAG TGG ATA GGG TCT ATC GAC CCG TAT TAT
GGG GAC ACA AAA TAC GCG CAG AAA TTC AAG GGG AAG GCC ACC CTG ACC
GTA GAT AAA GCT AGT TCT ACT GCG TAC TTG CAA CTG AAA AGC CTC ACT
TCT GAG GAC TCT GCC GTC TAC TAC TGT GCT CGG CGA ATG ATA ACG ACG
GGG GAC TGG TAT TTC GAT GTT TGG GGT ACA GGG ACT ACG GTG ACT GTC
AGT AGC tccgga
CD5-34H2L-3052 CAR
(SEQ ID NO: 4)
ggatccGAGGTTAAACTCGTGGAGAGCGGTGCCGAACTCGTCCGAAGTGGTGCTTC
CGTTAAACTCAGTTGTGCCGCGTCAGGATTTAACATAAAAGATTACTACATTCAC
TGGGTCAAACAGCGCCCGGAGCAGGGGCTTGAATGGATCGGGTGGATTGATCCT
GAAAACGGGCGCACCGAATATGCTCCCAAGTTCCAGGGCAAAGCTACTATGACC
GCTGACACCTCTAGTAACACTGCCTACCTGCAGTTGAGCTCTCTTACGTCTGAGG
ATACCGCTGTGTACTACTGTAATAACGGAAATTATGTACGACACTATTACTTCGA
CTACTGGGGGCAGGGCACTACTGTGACTGTATCTAGCggtggcggagggagcggcggtggagg
aagcggaggcggaggttccGATTGGCTCACACAATCCCCTGCAATCCTGAGTGCATCTCCA
GGCGAGAAAGTAACTATGACTTGCAGAGCTATAAGCTCTGTGTCCTACATGCACT
GGTATCAGCAGAAGCCAGGTTCTTCCCCGAAGCCGTGGATATATGCTACAAGCA
ATTTGGCATCCGGTGTTCCCGCCCGGTTTAGTGGCTCCGGTTCTGGGACAAGTTA
CTCCCTCACGATCAGCAGGGTTGAAGCCGAGGACGCTGCCACTTACTATTGCCA
ACAGTGGTCAAGTAACCCCAGGACTTTCGGGGGAGGAACTAAACTTGAAATCAA
Atctaga
CD5-34L2H-3053 CAR
(SEQ ID NO: 5)
Ggatcc GAT TGG CTC ACA CAA TCC CCT GCA ATC CTG AGT GCA TCT CCA GGC
GAG AAA GTA ACT ATG ACT TGC AGA GCT ATA AGC TCT GTG TCC TAC ATG
CAC TGG TAT CAG CAG AAG CCA GGT TCT TCC CCG AAG CCG TGG ATA TAT
GCT ACA AGC AAT TTG GCA TCC GGT GTT CCC GCC CGG TTT AGT GGC TCC
GGT TCT GGG ACA AGT TAC TCC CTC ACG ATC AGC AGG GTT GAA GCC GAG
GAC GCT GCC ACT TAC TAT TGC CAA CAG TGG TCA AGT AAC CCC AGG ACT
TTC GGG GGA GGA ACT AAA CTT GAA ATC AAA
Ggtggcggagggagcggcggtggaggaagcggaggcggaggttcc GAG GTT AAA CTC GTG GAG AGC
GGT GCC GAA CTC GTC CGA AGT GGT GCT TCC GTT AAA CTC AGT TGT GCC
GCG TCA GGA TTT AAC ATA AAA GAT TAC TAC ATT CAC TGG GTC AAA CAG
CGC CCG GAG CAG GGG CTT GAA TGG ATC GGG TGG ATT GAT CCT GAA AAC
GGG CGC ACC GAA TAT GCT CCC AAG TTC CAG GGC AAA GCT ACT ATG ACC
GCT GAC ACC TCT AGT AAC ACT GCC TAC CTG CAG TTG AGC TCT CTT ACG
TCT GAG GAT ACC GCT GTG TAC TAC TGT AAT AAC GGA AAT TAT GTA CGA
CAC TAT TAC TTC GAC TAC TGG GGG CAG GGC ACT ACT GTG ACT GTA TCT
AGC tCTAGA
CD5-17H2L-3054 CAR
(SEQ ID NO: 6)
ggatccGAGGTCCAACTCGTAGAATCAGGTCCCGGATTGGTGCAACCATCCCAGAG
CCTCTCTATTACATGCACGGTCTCTGGATTTAGTCTGACCAATTACGATGTGCATT
GGGTGCGCCAGTCTCCCGGCAAGGGGTTGGAATGGCTTGGCGTTATATGGAACT
ACGGAAATACAGACTATAACGCCGCGTTTATCTCTCGGCTGAGTATACGGAAAG
ACAGTAGTAAATCCCAGGTCTTTTTTACGATGTCATCCCTGCAAACGCCAGATAC
CGCAATATATTACTGCGCCAGGAACCACGGTGATGGTTATTATAATTGGTACTTC
GATGTGTGGGGTACTGGCACTACAGTCACAGTATCTTCAggtggcggagggagcggcggtgg
aggaagcggaggcggaggttccAACATTGTACTGACGCAAAGCCCCTCATCTTTGTCTGAGT
CACTCGGCGGCAAAGTAACCATCACATGCAAGGCCAGTCAAGACATCAATAAAT
ATATTGCTTGGTATCAGTATAAACCCGGCAAGGGGCCGCGACTGCTGATTCACTA
CACGAGTACCTTGCAACCGGGCATTCCGAGCCGATTTAGTGGCAGTGGCTCAGG
TCGCGATTACTCATTCTCAATAAGTAATCTCGAACCGGAAGACATAGCTACTTAT
TATTGCTTGCAGTACGATAATTTGTGGACCTTCGGGGGTGGTACAAAGTTGGAAA
TAAAGtctaga
CD8 Transmembrane domain nucleic acid sequence (SEQ ID NO: 7):
atctacatct gggcgccctt ggccgggact tgtggggtcc ttctcctgtc actggttatc
accctttact gc
CD8 Transmembrane domain amino acid sequence (SEQ ID NO: 8):
IYIWAPLAGTCGVLLLSLVITLYC
CD8 hinge domain nucleic acid sequence (SEQ ID NO: 9):
accacgacgc cagcgcegcg accaccaaca ccggcgccca ccatcgcgtc gcagcccctg
tccctgcgcc cagaggcgtg ccggccagcg gcggggggcg cagtgcacac gagggggctg
gacttcgcct gtgat
CD8 hinge domain amino acid sequence (SEQ ID NO: 10);
TTTPAPRPPTPAPTIASQPLSLRPEACRPAAGGAVHTRGLDFACD
4-1BB nucleic acid sequence
(SEQ ID NO: 11)
aaacggggca gaaagaaact cctgtatata ttcaaacaac catttatgag accagtacaa
actactcaag aggaagatgg ctgtagctgc cgatttccag aagaagaaga aggaggatgt
gaactg
CD3-zeta nucleic acid sequence
(SEQ ID NO: 12)
agagtgaagt tcagcaggag cgcagacgcc cccgcgtaca agcagggcca gaaccagctc
tataacgagc tcaatctagg acgaagagag gagtacgatg ttttggacaa gagacgtggc
cgggaccctg agatgggggg aaagccgaga aggaagaacc ctcaggaagg cctgtacaat
gaactgcaga aagataagat ggcggaggcc tacagtgaga ttgggatgaa aggcgagcgc
cggaggggca aggggcacga tggcctttac cagggtctca gtacagccac caaggacacc
tacgacgccc ttcacatgca ggccctgccc cctcgc
4-1BB amino acid sequence (SEQ ID NO: 13):
KRGRKKLLYIFKQPFMRPVQTTQEEDGCSCRFPEEEEGGCEL
CD3-zeta amino acid sequence (SEQ ID NO: 14):
RVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
CD5-17L2H-3045 CAR amino acid sequence
(SEQ ID NO: 15)
MALPVTALLLPLALLLHAARPGSNIVLTQSPSSLSESLGGKVTITCKASQDINKYIAW
YQYKPGKGPRLLIHYTSTLQPGIPSRFSGSGSGRDYSFSISNLEPEDIATYYCLQYDNL
WTFGGGTKLEIKGGGGSGGGGSGGGGSEVQLVESGPGLVQPSQSLSITCTVSGFSLT
NYDVHWVRQSPGKGLEWLGVIWNYGNTDYNAAFISRLSIRKDSSKSQVFFTMSSLQ
TPDTAIYYCARNHGDGYYNWYFDVWGTGTTVTVSSSRTTTPAPRPPTPAPTIASQPL
SLRPEACRPAAGGAVHTRGLDFACDIYIWAPLAGTCGVLLLSLVITLYCHMKRGRK
KLLYIFKQPFMRPVQTTQEEDGCSCRFPEEEEGGCELTSRVKFSRSADAPAYQQGQN
QLYNELNLGRREEYDVLDKRRGRDPEMGGKPRRKNPQEGLYNELQKDKMAEAYSE
IGMKGERRRGKGHDGLYQGLSTATKDTYDALHMQALPPR
CD5-17H2L-3054 CAR amino acid sequence
(SEQ ID NO: 16)
MALPVTALLLPLALLLHAARPGSEVQLVESGPGLVQPSQSLSITCTVSGFSLTNYDVH
WVRQSPGKGLEWLGVIWNYGNTDYNAAFISRLSIRKDSSKSQVFFTMSSLQTPDTAI
YYCARNHGDGYYNWYFDVWGTGTTVTVSSGGGGSGGGGSGGGGSNIVLTQSPSSL
SESLGGKVTITCKASQDINKYIAWYQYKPGKGPRLLIHYTSTLQPGIPSRFSGSGSGR
DYSFSISNLEPEDIATYYCLQYDNLWTFGGGTKLEIKSRTTTPAPRPPTPAPTIASQPLS
LRPEACRPAAGGAVHTRGLDFACDIYIWAPLAGTCGVLLLSLVITLYCHMKRGRKK
LLYIFKQPFMRPVQTTQEEDGCCRFPEEEEGGCELTSRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
CD5-17L2H-3045 scFv amino acid sequence
(SEQ ID NO: 17)
NIVLTQSPSSLSESLGGKVTITCKASQDINKYIAWYQYKPGKGPRLLIHYTSTLQPGIP
SRFSGSGSGRDYSFSISNLEPEDIATYYCLQYDNLWTFGGGTKLEIKGGGGSGGGGS
GGGGSEVQLVESGPGLVQPSQSLSITCTVSGFSLTNYDVHWVRQSPGKGLEWLGVI
WNYGNTDYNAAFISRLSIRKDSSKSQVFFTMSSLQTPDTAIYYCARNHGDGYYNWY
FDVWGTGTTVTVSS
CD5-17H2L-3054 scFv amino acid sequence
(SEQ ID NO: 18)
EVQLVESGPGLVQPSQSLSITCTVSGFSLTNYDVHWVRQSPGKGLEWLGVIWNYGN
TDYNAAFISRLSIRKDSSKSQVFFTMSSLQTPDTAIYYCARNHGDGYYNWYFDVWG
TGTTVTVSSGGGGSGGGGSGGGGSNIVLTQSPSSLSESLGGKVTITCKASQDINKYIA
WYQYKPGKGPRLLIHYTSTLQPGIPSRFSGSGSGRDYSFSISNLEPEDIATYYCLQYDN
LWTFGGGTKLEIK
CD5-17 VH amino acid sequence
(SEQ ID NO: 19)
EVQLVESGPGLVQPSQSLSITCTVSGFSLTNYDVHWVRQSPGKGLEWLGVIWNYGN
TDYNAAFISRLSIRKDSSKSQVFFTMSSLQTPDTAIYYCARNHGDGYYNWYFDVWG
TGTTVTVSS
CD5-17 VL amino acid sequence
(SEQ ID NO: 20)
NIVLTQSPSSLSESLGGKVTITCKASQDINKYIAWYQYKPGKGPRLLIHYTSTLQPGIP
SRFSGSGSGRDYSFSISNLEPEDIATYYCLQYDNLWTFGGGTKLEIK
CD5-9H2L-3048 CAR amino acid sequence
(SEQ ID NO: 21)
MALPVTALLLPLALLLHAARPGSQVQLKESGPELEKPGASVKISCKASGYSFTAYSM
NWVKQNNGMSLEWIGSIDPYYGDTKYAQKFKGKATLTVDKASSTAYLQLKSLTSE
DSAVYYCARRMITTGDWYFDVWGTGTTVTVSSGGGGSGGGGSGGGGSHIVLTQSP
SSLSASLGDRVTISCRASQDISTYLNWYQQKPDGTVKLLIFYTSRLHAGVPSRFSGSG
SGTHHSLTISNLEQEDIATYFCQQGNSLPFTFGSGTKLEIKSGTTTPAPRPPTPAPTIAS
QPLSLRPEACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIG
MKGERRRGKGHDGLYQGLSTATKDTYDALHMQALPPR
CD5-9L2H-3049 CAR amino acid sequence
(SEQ ID NO: 22)
MALPVTALLLPLALLLHAARPGSHIVLTQSPSSLSASLGDRVTISCRASQDISTYLNW
YQQKPDGTVKLLIFYTSRLHAGVPSRFSGSGSGTHHSLTISNLEQEDIATYFCQQGNS
LPFTFGSGTKLEIKGGGGSGGGGSGGGGSQVQLKESGPELEKPGASVKISCKASGYSF
TAYSMNWVKQNNGMSLEWIGSIDPYYGDTKYAQKFKGKATLTVDKASSTAYLQLK
SLTSEDSAVYYCARRMITTGDWYFDVWGTGTTVTVSSSGTTTPAPRPPTPAPTIASQP
LSLRPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYKQGQNQLYN
ELNLGRREEYDVLDKRRGRDPEMGGKPRRKNPQEGLYNELQKDKMAEAYSEIGMK
GERRRGKGHDGLYQGLSTATKDTYDALHMQALPPR
CD5-9H2L-3048 scFv amino acid sequence
(SEQ ID NO: 23)
GSQVQLKESGPELEKPGASVKISCKASGYSFTAYSMNWVKQNNGMSLEWIGSIDPY
YGDTKYAQKFKGKATLTVDKASSTAYLQLKSLTSEDSAVYYCARRMITTGDWYFD
VWGTGTTVTVSSGGGGSGGGGSGGGGSHIVLTQSPSSLSASLGDRVTISCRASQDIST
YLNWYQQKPDGTVKLLIFYTSRLHAGVPSRFSGSGSGTHHSLTISNLEQEDIATYFCQ
QGNSLPFTFGSGTKLEIKSG
CD5-9L2H-3049 scFv amino acid sequence
(SEQ ID NO: 24)
HIVLTQSPSSLSASLGDRVTISCRASQDISTYLNWYQQKPDGTVKLLIFYTSRLHAGV
PSRFSGSGSGTHHSLTISNLEQEDIATYFCQQGNSLPFTFGSGTKLEIKGGGGSGGGGS
GGGGSQVQLKESGPELEKPGASVKISCKASGYSFTAYSMNWVKQNNGMSLEWIGSI
DPYYGDTKYAQKFKGKATLTVDKASSTAYLQLKSLTSEDSAVYYCARRMITTGDW
YFDVWGTGTTVTVSS
CD5-9 VH amino acid sequence
(SEQ ID NO: 25)
QVQLKESGPELEKPGASVKISCKASGYSFTAYSMNWVKQNNGMSLEWIGSIDPYYG
DTKYAQKFKGKATLTVDKASSTAYLQLKSLTSEDSAVYYCARRMITTGDWYFDVW
GTGTTVTVSS
CD5-9 VL amino acid sequence
(SEQ ID NO: 26)
HIVLTQSPSSLSASLGDRVTISCRASQDISTYLNWYQQKPDGTVKLLIFYTSRLHAGV
PSRFSGSGSGTHHSLTISNLEQEDIATYFCQQGNSLPFTFGSGTKLEIK
CD5-9 HCDR1
(SEQ ID NO: 27)
AYSMN
CD5-9 HCDR2
(SEQ ID NO: 28)
SIDPYYGDTKYAQKFKG
CD5-9 HCDR3
(SEQ ID NO: 29)
RMITTGDWYFDV
CD5-9 LCDR1
(SEQ ID NO: 30)
RASQDISTYLN
CD5-9 LCDR2
(SEQ ID NO: 31)
YTSRLHA
CD5-9 LCDR3
(SEQ ID NO: 32)
QQGNSLPFT
CD5-34H2L-3052 CAR amino acid sequence
(SEQ ID NO: 33)
MALPVTALLLPLALLLHAARPGSEVKLVESGAELVRSGASVKLSCAASGFNIKDYYI
HWVKQRPEQGLEWIGWIDPENGRTEYAPKFQGKATMTADTSSNTAYLQLSSLTSED
TAVYYCNNGNYVRHYYFDYWGQGTTVTVSSGGGGSGGGGSGGGGSDWLTQSPAI
LSASPGEKVTMTCRAISSVSYMHWYQQKPGSSPKPWIYATSNLASGVPARFSGSGSG
TSYSLTISRVEAEDAATYYCQQWSSNPRTFGGGTKLEIKSRTTTPAPRPPTPAPTIASQ
PLSLRPEACRPAAGGAVHTRGLDFACDIYIWAPLAGTCGVLLLSLVITLYCHMKRGR
KKLLYIFKQPFMRPVQTTQEEDGCSCRFPEEEEGGCELTSRVKFSRSADAPAYQQGQ
NQLYNELNLGRREEYDVLDKRRGRDPEMGGKPRRKNPQEGLYNELQKDKMAEAY
SEIGMKGERRRGKGHDGLYQGLSTATKDTYDALHMQALPPR
CD5-34L2H-3053 CAR amino acid sequence
(SEQ ID NO: 34)
MALPVTALLLPLALLLHAARPGSDWLTQSPAILSASPGEKVTMTCRAISSVSYMHW
YQQKPGSSPKPWIYATSNLASGVPARFSGSGSGTSYSLTISRVEAEDAATYYCQQWS
SNPRTFGGGTKLEIKGGGGSGGGGSGGGGSEVKLVESGAELVRSGASVKLSCAASG
FNIKDYYIHWVKQRPEQGLEWIGWIDPENGRTEYAPKFQGKATMTADTSSNTAYLQ
LSSLTSEDTAVYYCNNGNYVRHYYFDYWGQGTTVTVSSSRTTTPAPRPPTPAPTIAS
QPLSLRPEACRPAAGGAVHTRGLDFACDIYIWAPLAGTCGVLLLSLVITLYCHMKRG
RKKLLYIFKQPFMRPVQTTQEEDGCSCRFPEEEEGGCELTSRVKFSRSADAPAYQQG
QNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CD5-34H2L-3052 scFv amino acid sequence
(SEQ ID NO: 35)
EVKLVESGAELVRSGASVKLSCAASGFNIKDYYIHWVKQRPEQGLEWIGWIDPENG
RTEYAPKFQGKATMTADTSSNTAYLQLSSLTSEDTAVYYCNNGNYVRHYYFDYWG
QGTTVTVSSGGGGGGGGSGGGGSDWLTQSPAILSASPGEKVTMTCRAISSVSYMH
WYQQKPGSSPKPWIYATSNLASGVPARFSGSGSGTSYSLTISRVEAEDAATYYCQQ
WSSNPRTFGGGTKLEIK
CD5-34L2H-3053 scFv amino acid sequence
(SEQ ID NO: 36)
DWLTQSPAILSASPGEKVTMTCRAISSVSYMHWYQQKPGSSPKPWIYATSNLASGVP
ARFSGSGSGTSYSLTISRVEAEDAATYYCQQWSSNPRTFGGGTKLEIKGGGGSGGGG
SGGGGSEVKLVESGAELVRSGASVKLSCAASGFNIKDYYIHWVKQRPEQGLEWIGW
IDPENGRTEYAPKFQGKATMTADTSSNTAYLQLSSLTSEDTAVYYCNNGNYVRHYY
FDYWGQGTTVTVSS
CD5-34 VH amino acid sequence
(SEQ ID NO: 37)
EVKLVESGAELVRSGASVKLSCAASGFNIKDYYIHWVKQRPEQGLEWIGWIDPENG
RTEYAPKFQGKATMTADTSSNTAYLQLSSLTSEDTAVYYCNNGNYVRHYYFDYWG
QGTTVTVSS
CD5-34 VL amino acid sequence
(SEQ ID NO: 38)
DWLTQSPAILSASPGEKVTMTCRAISSVSYMHWYQQKPGSSPKPWIYATSNLASGVP
ARFSGSGSGTSYSLTISRVEAEDAATYYCQQWSSNPRTFGGGTKLEIKSR
CD5-34 HCDR1
(SEQ ID NO: 39)
DYYIH
CD5-34 HCDR2
(SEQ ID NO: 40)
WIDPENGRTEYAPKFQG
CD5-34 HCDR3
(SEQ ID NO: 41)
GNYVRHYYFDY
CD5-34 LCDR1
(SEQ ID NO: 42)
RAISSVSYMH
CD5-34 LCDR2
(SEQ ID NO: 43)
ATSNLAS
CD5-34 LCDR3
(SEQ ID NO: 44)
QQWSSNPRT

### D. Methods of Producing Genetically Modified Immune Cells

The present disclosure provides methods for producing or generating a modified immune cell or precursor thereof (e.g., a T cell), e.g., for adoptive immunotherapy. The cells generally are engineered by introducing into the cell one or more nucleic acids encoding the CAR and/or one or more agents (e.g. nucleic acids) that knock-out, mutate, or otherwise modify the endogenous CD5.

In certain embodiments, the immune cell or precursor cell thereof is a T cell. In certain embodiments, the T cell is human T cell. In certain embodiments, T cell is an autologous T cell.

In some embodiments, a nucleic acid molecule encoding the CAR and/one or more nucleic acids that knock-out or mutate the endogenous CD5 are introduced into a cell by an expression vector. Expression vectors comprising a nucleic acid sequence encoding a CAR of the are also provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid encoding a CAR and/or the one or more nucleic acid molecules that knock-out or mutate the endogenous CD5 are introduced into the cell via viral transduction. In some embodiments, the viral transduction is performed in vivo.

Examples of in vivo transduction to introduce a heterologous nucleic acid molecule are known and any such method can be utilized. The in vivo transduction can be used to introduce the nucleic acid molecule encoding the CAR and/or the one or more nucleic acid molecules that knock-out or mutate the endogenous CD5.

In certain embodiments, the viral transduction comprises contacting the immune or precursor cell with a viral vector comprising the nucleic acid encoding an exogenous CAR and/or the one or more nucleic acid molecules that knock-out the endogenous CD5. In certain embodiments, the viral vector is an adeno-associated viral (AAV) vector. In certain embodiments, the AAV vector comprises a 5′ ITR and a 3′ITR derived from AAV6. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the CAR in the host cell. In some embodiments, the adenovirus genome is a 36 kb, linear, double stranded DNA, where a foreign DNA sequence (e.g., a nucleic acid encoding an exogenous CAR) may be inserted to substitute large pieces of adenoviral DNA in order to make the expression vector (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding a CAR) into the viral genome at certain locations to produce a virus that is replication defective. Though the retroviral vectors are able to infect a broad variety of cell types, integration and stable expression of the CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a CAR (see, e.g., U.S. Pat. No. 5,994,136).

Expression vectors including a nucleic acid of the present disclosure can be introduced into a host cell by any method known to persons skilled in the art, such as, but not limited to transfection, transduction, and electroporation. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

Embodiments provided for herein also provide genetically engineered cells (e.g. cells with a mutated or disrupted CD5 gene), which include and stably express a CAR of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells.

Modified cells (e.g., comprising (expressing) a CAR and wherein endogenous CD5 has been disrupted and/or knocked out) may be produced by stably transfecting host cells with an expression vector including a nucleic acid of the present disclosure. These can also be produced in vivo by administering a viral particle that can infect such cells in vivo to produce the modified cells in vivo. Examples of in vivo transduction to introduce a heterologous nucleic acid molecule can be found, for example, in U.S. Application No., which is hereby incorporated by reference in its entirety. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a CAR of the present disclosure may be expanded ex vivo or expanded in vivo by administering other therapeutics that can stimulate the expansion of the modified cell.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In some embodiments, the nucleic acids introduced into the host cell are RNA. In some embodiments, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA. 5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a CAR of the present disclosure will be RNA, e.g., in vitro synthesized RNA. Methods for in vitro synthesis of RNA are known in the art; any known method can be used to synthesize RNA comprising a sequence encoding a CAR. Methods for introducing RNA into a host cell are known in the art. See, e.g., Zhao et al. Cancer Res. (2010) 15: 9053. Introducing RNA comprising a nucleotide sequence encoding a CAR into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

RNA transfection methods can be used without a vector, such as a plasmid or a virus. An RNA transgene, such as those encoding for the CAR can be delivered to a lymphocyte and expressed therein following a cell activation, as a minimal expressing cassette without the need for any additional viral sequences. Cloning of cells may not be necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the exogenous receptor (e.g., CAR). In some embodiments, the cells (e.g. T cells) can be incubated or cultivated prior to, during or subsequent to the introduction of the nucleic acid molecule encoding the exogenous receptor, such as prior to, during or subsequent to the transduction of the cells with a viral vector (e.g. lentiviral vector) encoding the exogenous receptor.

### E. Sources of Immune Cells

In some embodiments, a source of immune cells is obtained from a subject (e.g. for ex vivo manipulation). Sources of cells manipulation may also include, e.g., autologous or allogeneic donor blood, cord blood, or bone marrow. For example the source of immune cells may be from the subject to be treated with the modified immune cells, e.g., the subject's blood, the subject's cord blood, or the subject's bone marrow. Non-limiting examples of subjects include humans, dogs, cats, mice, rats, and transgenic species thereof. Preferably, the subject is a human.

The cells may also be created by transducing the cells in vivo, such as, but not limited to, by the methods described herein. In some embodiments, the viral transduction can be directed to certain immune cells by incorporating a targeting moiety into the viral particle.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, if performed ex vivo, the methods include isolating immune cells from the subject, preparing, processing, culturing, and/or engineering/modifying them. In some embodiments, preparation of the engineered cells includes one or more culture and/or preparation steps. The cells for engineering/modifying as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD1 1b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering/modification. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In certain embodiments, the modified cells are expanded without any stimulating agents. In certain embodiments, the modified cells are expanded in vivo.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### F. Expansion of T Cells

In certain embodiments, the modified T cells provided for herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In some embodiments embodiment, the modified T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In some embodiments, the cells are cryopreserved or the expanded cells are cryopreserved. The cryopreserved T cells are thawed prior to introducing nucleic acids into the T cell.

In another embodiment, the method comprises isolating T cells and expanding the T cells. In another embodiment, the methods further comprises cryopreserving the T cells prior to expansion. In yet another embodiment, the cryopreserved T cells are thawed for electroporation with the RNA encoding the chimeric membrane protein. These introductions can be done before or after the cell is modified to mutate or otherwise disrupt the CD5 gene.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### G. Pharmaceutical Compositions and Formulations

Also provided herein are populations of immune cells (e.g. CAR T cells wherein CD5 has been disrupted and/or knocked out) and compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for intravenous, intraperitoneal, subcutaneous, pulmonary, transdermal, intramuscular, intranasal, buccal, sublingual, or suppository administration. In some embodiments, the pharmaceutical compositions are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells or viral particles in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present application describes various embodiments, it should be understood by those skilled in the art that various changes may be made and equivalents may be substituted without departing from the true spirit and scope of the embodiments. It will be readily apparent to those skilled in the art that other suitable modifications and adaptations of the compositions and methods described herein may be made using suitable equivalents without departing from the scope of the embodiments disclosed herein. In addition, many modifications may be made to adapt a particular situation, material, composition of matter, process, process step or steps, to the objective, spirit and scope of the embodiments provided for herein. All such modifications are intended to be within the scope of the embodiments provided for herein and claims appended hereto. Having now described certain embodiments in detail, the same will be more clearly understood by reference to the following examples, which are included for purposes of illustration only and are not intended to be limiting.

### Experimental Examples

The embodiments are now described with reference to the following Examples. These Examples are provided for the purpose of illustration only, and the embodiments are not limited to these Examples, but rather encompasses all variations that are evident as a result of the teachings provided herein.

The materials and methods employed in these experiments are now described.

Small guide RNAs (sgRNAs) were designed to target CD5 and synthesized using the GeneArt Precision sgRNA synthesis kit. One sgRNA comprised the sequence CGGCTCAGCTGGTATGACCC (SEQ ID NO: 45). Cas9 expression plasmid (pGEM-Cas9) was amplified and linearized. Cas9 RNA was synthesized using the mMessage mMachine T7 Ultra kit. CRISPR editing was performed in Jurkat cells: CD5 sgRNAs and Cas9 were transfected into Jurkat cells by electroporation. Expression of CD5 on Jurkat cells was detected by flow cytometry and the most effective CD5 sgRNAs were determined. CRISPR editing was then performed in primary human T cells using the most efficient CD5 sgRNA: the chosen sgRNA and Cas9 RNA were electroporated into primary human T cells. CD5 expression was detected on the primary human T cells by flow cytometry to validate the knock-out/editing efficiency.

Specifically, fresh CD4/CD8 T cells were obtained and incubated with dynabeads on day 0. On day 4, cells were de-beaded then electroporated with Cas9 and sgRNA. Conditioned media (TCM (X-vivo15, human serum 5%, Glutamine), IL-7 10 ng/ml, and IL-15 10 ng/ml) was added to the cells. On day 6, cells were transduced with CAR lentivirus. On day 9, CAR expression was assessed. Cells were fed to 0.8e6/ml and frozen when volume <300 fl.

CAR constructs: All constructs were generated using the lentiviral pTRPE 4-1BB CD3zeta backbone. CD5 CARs were constructed using scFvs from antibody sequences published in WO 2010/022737 A1, contents of which are incorporated by reference in their entirety herein.

The results of the experiments are now described.

### Example 1

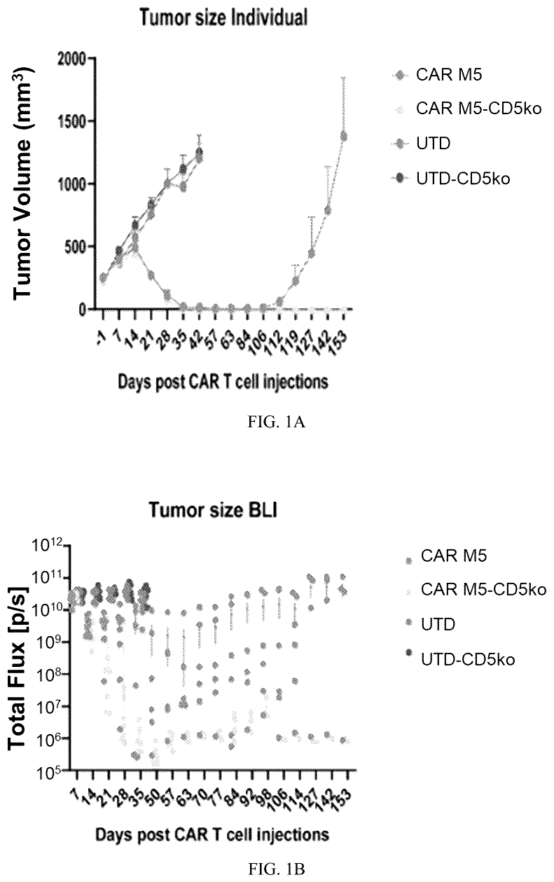

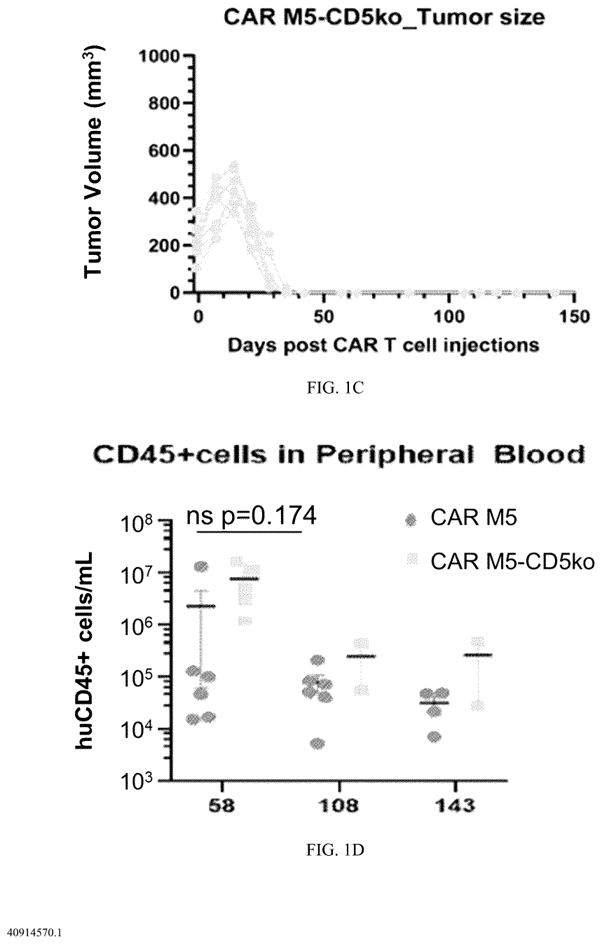

The efficacy of CD5 KO CAR T cells was tested in a solid cancer xenograft model. NOD SCID gamma chain −/− (NSG) mice were engrafted with the pancreatic cancer cell line ASPC1 that was previously transduced with luciferase. Cancer cells were injected subcutaneously with Matrigel. Tumors were measured by caliper and bioluminescence. At day 14 mice were randomized based on tumor burden to receive control untransduced T cells (UTD), CD5 knock out (KO) UTD, mock control anti-mesothelin CAR T cells or CD5 KO CAR T cells. Tumor burden was measured over time by caliper and luminescence. Caliper measurements showed complete remission in both groups but only CD5 KO CAR T maintained the remission long term, while control CAR T relapsed at about 3 months (FIG. 1A). Tumor burden by bioluminescence showed early and deep tumor clearance by CD5 KO CAR T as compared to mock control CAR T or UTD (FIG. 1). The in vivo expansion in the peripheral blood of T cells was measured in all groups using flow cytometry (FIG. 1C). Interestingly, CD5 KO CAR T showed higher expansion as compared to mock control CAR T. These results demonstrated that CD5 knock out enhanced the ability of CAR T to control solid cancer in a preclinical model of solid cancer (pancreatic cancer). Enhanced proliferation of CAR T cells was associated with this prolonged anti-tumor effect.

### Example 2

### CD5 Knock Out Enhanced the Anti-Tumor Activity of Chimeric Antigen Receptor T Cells.

Chimeric Antigen Receptor T cells (CAR T) have led to unprecedented clinical responses in relapsed or refractory B-cell (r/r) acute lymphoblastic leukemia (B-ALL), non-Hodgkin lymphomas (NHL), and multiple myeloma. Currently, all but one FDA-approved CAR T cell therapies target the B cell surface protein, CD19. While anti-CD19 CAR (CAR19) T (CART19) cell therapy has emerged as a standard approach for patients with relapsed and refractory B cell malignancies, there is still a significant fraction of patients who do not respond or eventually relapse. CAR T therapy has not yet been proven effective in hematological malignancies, including T cell lymphoma (TCL), T cell leukemias (T-ALL), acute myeloid leukemias (AML), an, even less so, in solid cancers (T-NHL). Moreover, CAR T therapy has not provided yet satisfying results in solid cancer due to the effect of the overwhelming immunosuppressive tumor microenvironment. Thus, there is a need in the art to enhance currently available CAR T products and there is also a need in the art to develop next-generation CAR T therapies to successfully treat additional neoplasms.

To this goal, the cysteine-rich scavenger receptor CD5 was studied herein. Indeed, CD5 is expressed in most T cells, and it is considered an attractive target for CAR T immunotherapy because of its dual role in malignant cells and normal T cells. In malignant cells, CD5 is an ideal target because it is expressed by ˜90% of T-NHL cells and by virtually all chronic lymphocytic and mantle cell lymphoma cells. On the other hand, CD5 is also highly expressed on the surface of normal T cells, where it functions as an inhibitory molecule on T cell receptor (TCR) mediated activation through several regulators such as SUP-1, CBL, and CBL-B. Indeed, the generation of an anti-CD5 CAR (CAR5) T cell product (CART5) would lead to the concomitant expression of CAR5 and CD5 on the surface of T cells, theoretically leading to the in-cis binding, subsequent exhaustion, and eventual fratricidal killing of transduced cells during manufacturing. Therefore, the hypothesis was that the genetic deletion of CD5 in engineered T cells can enhanced CAR T cell effector functions and avoid fratricide interactions.

As described herein, it was surprisingly found that a CAR T cell with a mutated CD5 gene (such as by utilizing CRISPR-Cas9 to mutate (knockout, “KO”)) enhanced the anti-tumor activity of CAR T cells in solid tumors by enhancement of CAR-mediated activation and proliferation. CD5 KO CAR-T enhanced anti-tumor efficacy of CAR T cells against solid tumors, such as, mesothelin+ pancreatic ductal adenocarcinoma (PDAC).

### Results

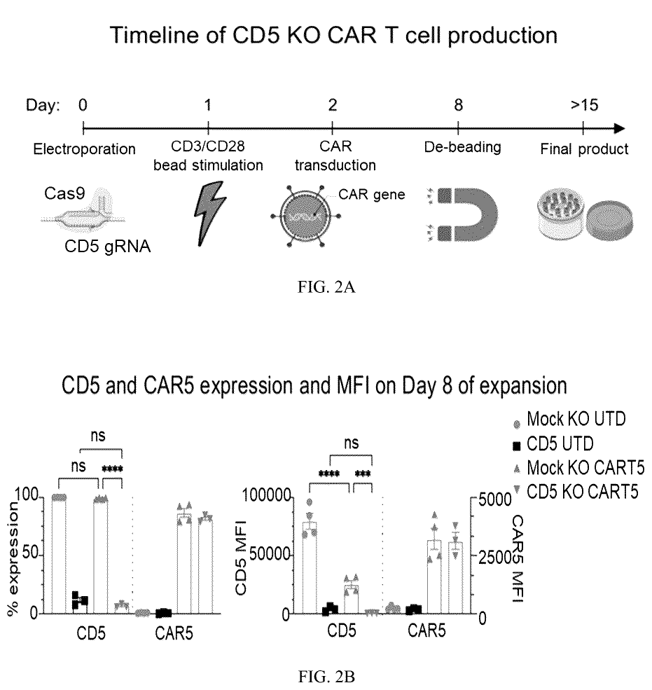

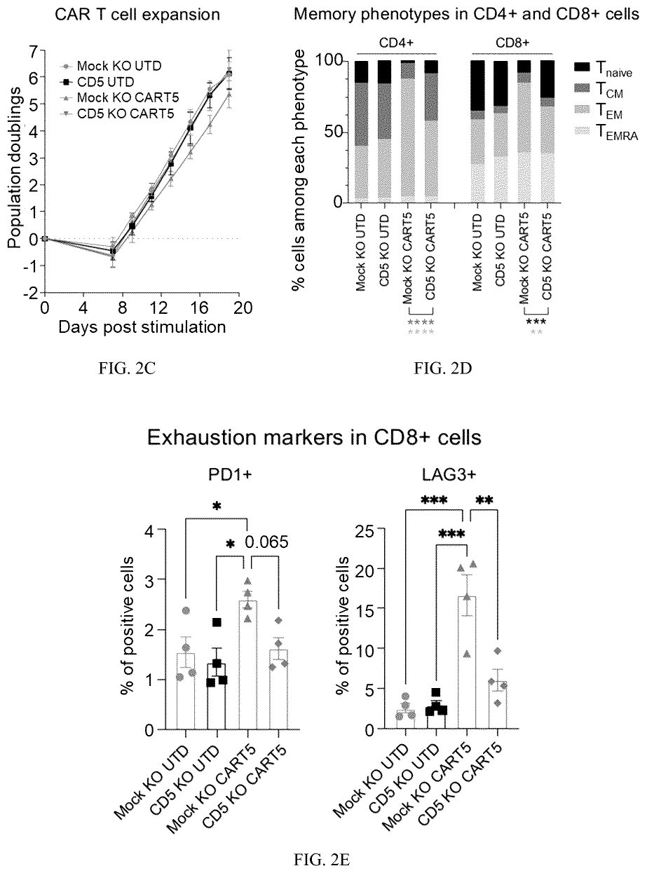

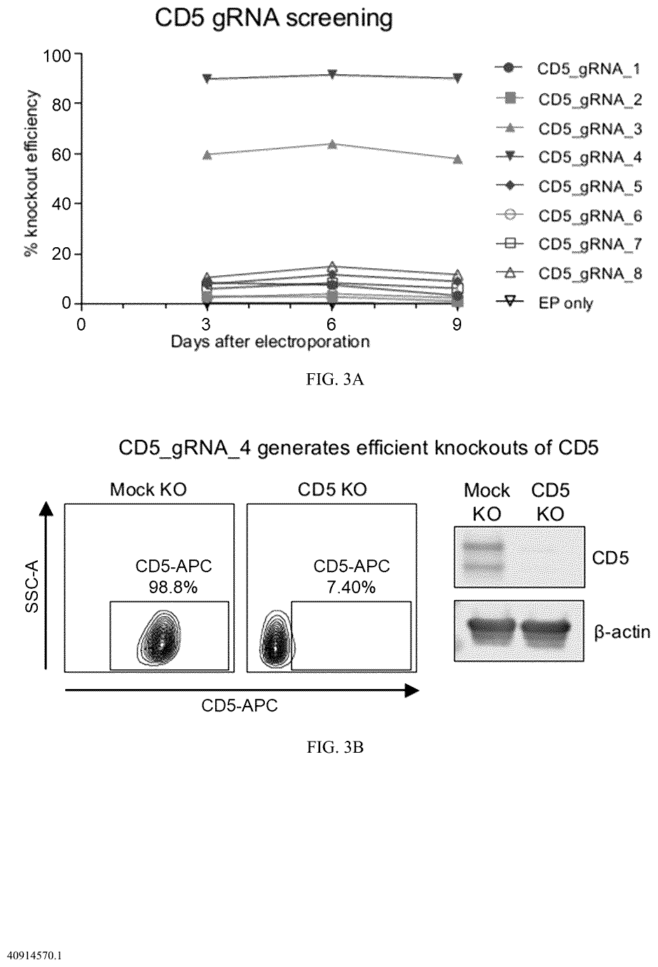

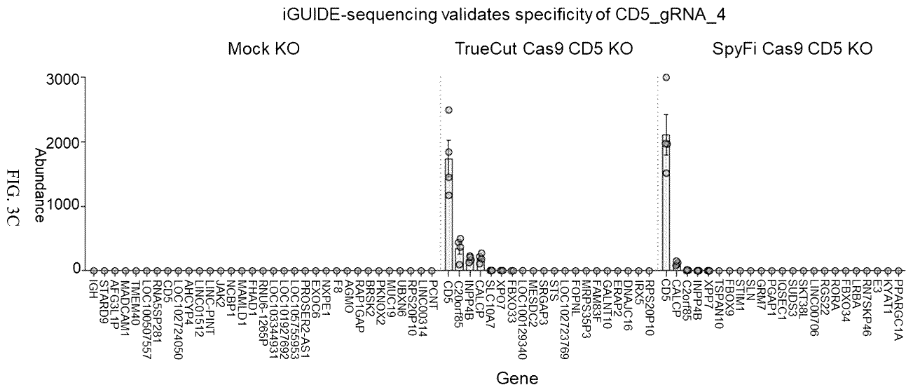

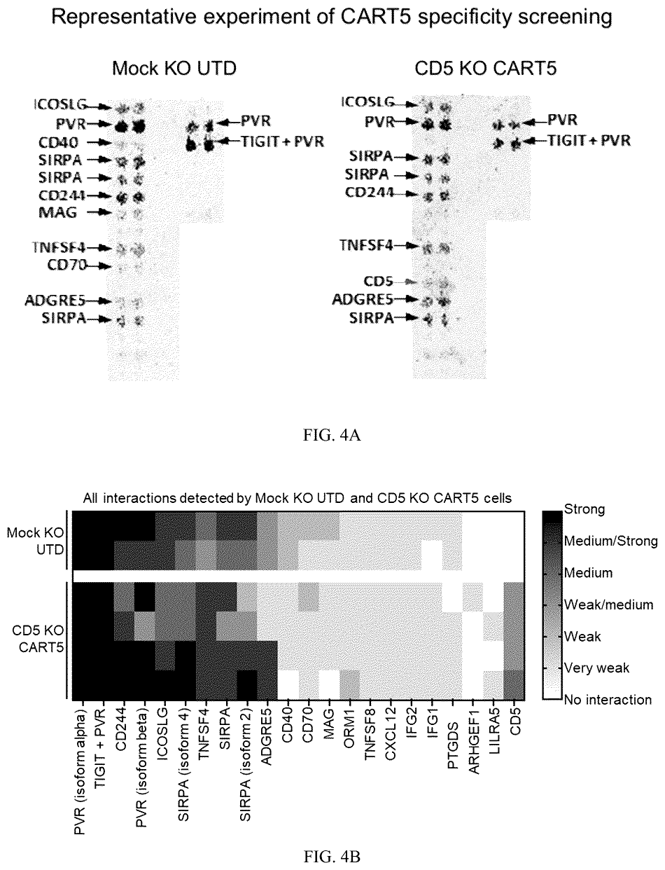

Genetic deletion of CD5 produced a more functionally active CART5 product: First, short guide RNAs targeting the exon 1 of CD5 were designed and optimized. To generate CD5 KO CART5 cells, a highly efficient CAR T manufacturing protocol was developed (FIG. 2A). A CD5 targeting single guide RNA (sgRNA) deleted CD5 in normal T cells using CRISPR-Cas9 (FIG. 3A; Table 1). A reproducibly efficient sequence was identified (>90% KO by flow cytometry and western blot) (FIG. 3B). Improved Genome-wide, Unbiased Identifications of Double Stranded Breaks Enabled by Sequencing known as iGUIDE-sequencing and analysis confirmed high on-targeted cleavage, with no off-target cleavage sites of concern detected when using this sequence within two separate donors (FIG. 3C). The top five off-target genes by TrueCut Cas9 or SpyFi Cas9 included CALCP, C20orf85, INPP4B, XPO7, and SLC10A7. All five potential genes demonstrated little to no expression in CD4+ or CD8+ naïve T cells as seen by bulk RNA sequencing analysis data (Database of Immune Cell Expression, Expression quantitative trait loci, and Epigenomics; DICE; https://dice-database.org). This indicated no obvious basis for concern regarding the location of off-target cleavage sites.

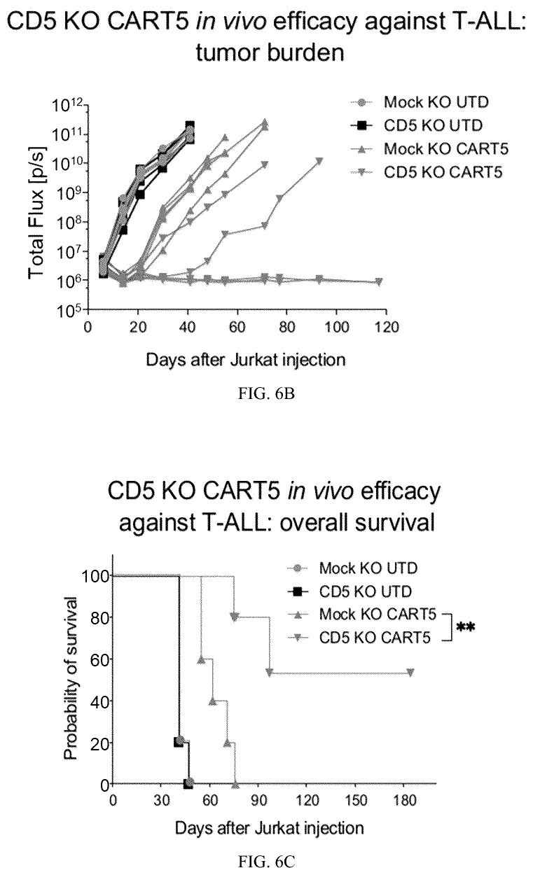

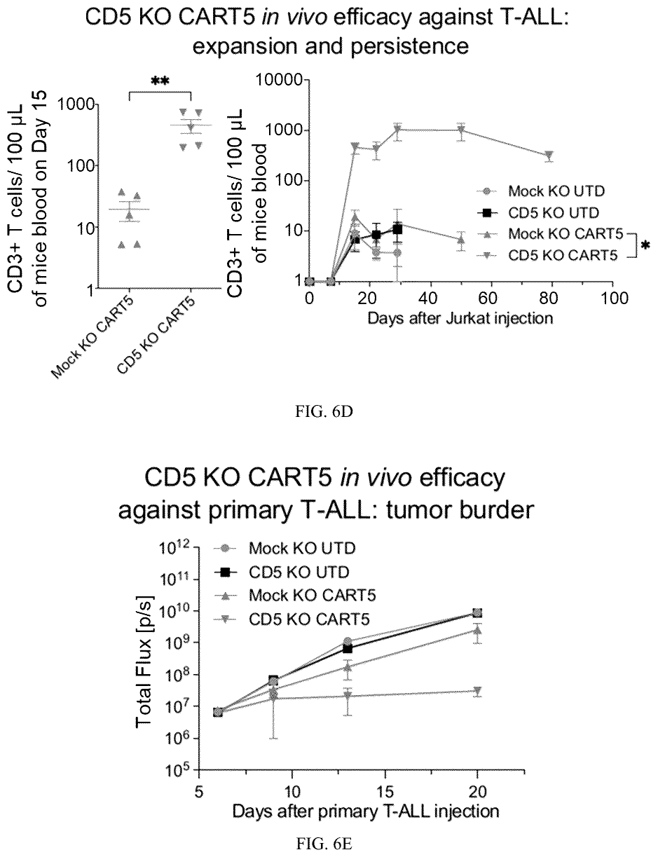

To target CD5 in TCL/T-ALL, six 4-1BB-costimulated CAR5 lentiviral constructs were designed and screened for high, medium, and low affinity for CD5. The lead CAR5 construct (high affinity, heavy-to-light chain orientation) was selected based on its superior anti-tumor function in vivo in NOD-SCID IL2Rg^{null} (NSG) mice engrafted with Jurkat T-ALL cell line. During CART5 manufacturing, CD5 KO reduced the CART-CART fratricide and led to higher expansions. Furthermore, at the end of manufacturing, CD5 KO CART5 had increased naïve T cells (CD8+ cells, p=0.0008) and reduced expression of activation/exhaustion markers (LAG3, p=0.0016) compared to Mock KO CART5. Mock KO CART5 were then compared to CD5 KO CART5 in vitro using several T-NHL/T-ALL, MCL, and AML models, including primary samples (Sezary cells, primary MCL cells, and CD5+ AML cells). Both Mock and CD5 KO CART5 were highly effective in killing CD5+ malignant cells, but CD5 KO CART5 showed enhanced proliferation upon activation. In two xenograft models of T-ALL (primary T-ALL and Jurkat), CD5 KO CART5 showed dramatically increased tumor control compared to Mock KO (FIG. 6B, median overall survival for Mock KO CART5=62 days vs. CD5 KO CART5=not reached, p=0.006, Mantel-Cox) (FIG. 6C). This enhanced anti-tumor effect was associated with increased expansion and persistence of CD5 KO CART5 in the peripheral blood (PB) compared to Mock KO CART5 (FIG. 6D).

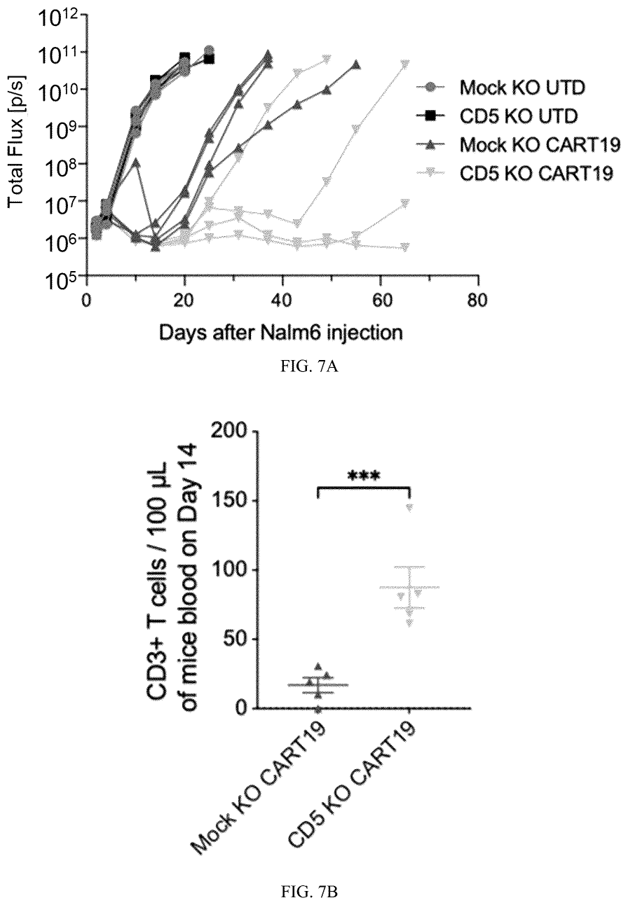

In order to exclude that the beneficial effect of CD5 deletion in the anti-CD5 CART model was only due to the elimination of fratricide, CD5 KO was tested in the standard anti-CD19 CART (CART19) model (4-1BB). CD5 was knocked out in CART19 cells and their function was tested in a CD19+ B-ALL xenograft model (NALM6). Remarkably, CD5 KO CART19 displayed significantly enhanced anti-leukemia activity and blood expansion compared to Mock KO (FIG. 7A-7B). It was then speculated that this platform could be applied to solid cancers that do not typically respond to current CART therapies. CD5 was deleted in anti-mesothelin CART cells (CARTmeso) using a clinically relevant CAR construct (M5 clone, U.S. Ser. No. 10/640,569B2, which is hereby incorporated by reference in its entirety).

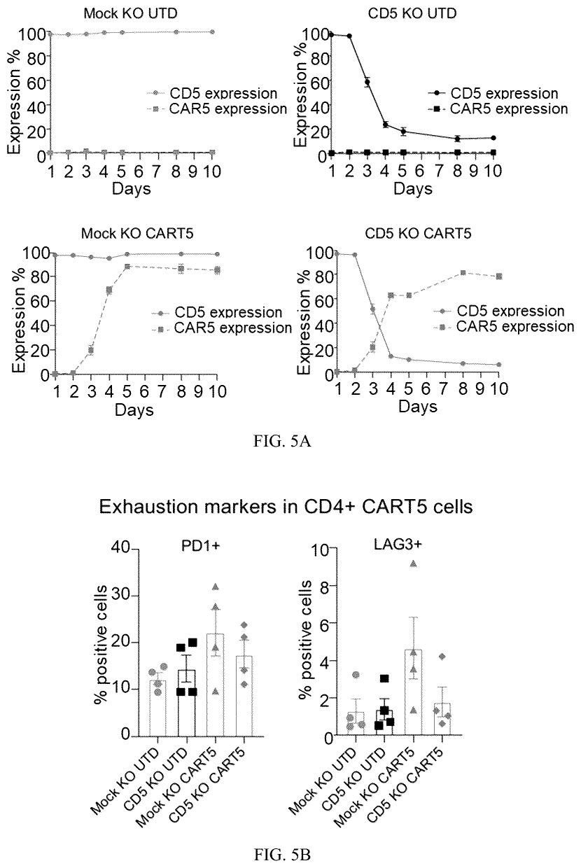

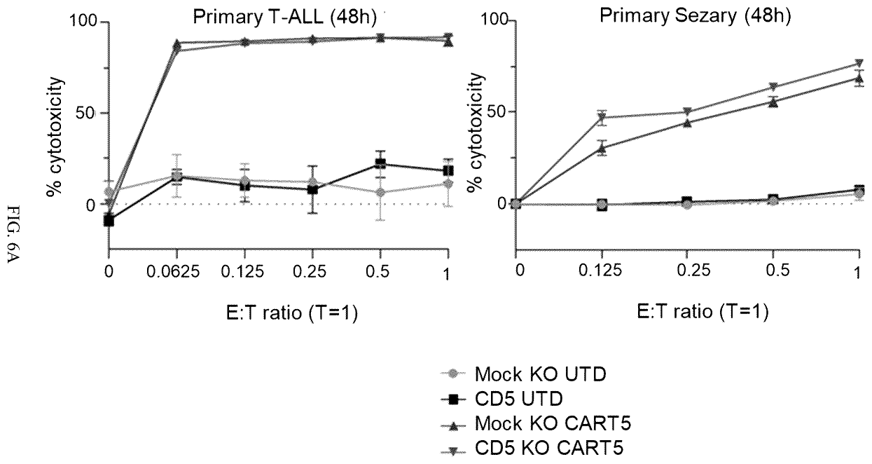

Interestingly, while Mock KO untransduced (UTD) cells showed similar CD5 expression in comparison to Mock KO CART5 cells, there was a significant reduction in the mean fluorescence intensity (MFI) of CD5. This suggested that CD5-CART5 engagement led to potential epitope masking on the surface of Mock KO CART5 cells (FIG. 2B; FIG. 5A). Notably, during manufacturing, the growth rate of Mock KO CART5 cells was reduced compared to CD5 KO CART5 (FIG. 2C). Moreover, at the end of manufacturing, CD5 KO CART5 cells were enriched of naïve T cells when compared to Mock KO CART5 cells (8.31%±1.77 vs. 1.17%±0.40 in CD4+ cells, ns; 25.75%±4.00 vs. 8.13%±2.79 in CD8+ cells, p=0.0008) and had lower effector memory T cells (53.18%±4.31 vs. 83.05%±3.72 in CD4+, p<0.0001; 33.18%±2.65 vs. 49.15%±5.36 in CD8+, p=0.00034) (FIG. 2D). Furthermore, CD5 KO CART5 cells presented lower expression of activation/exhaustion markers than Mock KO CART5 in CD8+ T cells (PD1: 1.62%±0.21 vs. 2.60%±0.17, ns; LAG3: 6.00%±1.36 vs. 16.64%±2.59, p=0.0016) (FIG. 2E; FIG. 5B). This data suggested that the CD5-CART5 engagement during manufacturing led to a final product which was enriched in exhausted T cells and that the genetic deletion of CD5 resulted in a more active and functional product.

CD5 KO CART5 T cells demonstrated greater efficacy than Mock KO CAR T5 cells in vivo: The efficacy of Mock KO CART5 and CD5 KO CART5 cells were evaluated in vitro against CD5-positive and CD5-negative cells. Both Mock KO CART5 and CD5 KO CART5 were highly effective in killing CD5-positive primary T-ALL, primary Sezary, and Jurkat cell line but demonstrated little to no cytotoxic effects against CD5-negative B-ALL cell line Nalm6 (FIG. 6A). To evaluate the in vivo efficacy of CD5 KO CART5 T cells, NSG mice were engrafted with Jurkat cells and administered engineered T cells or proper controls (FIG. 6B). CD5 KO CART5 cells dramatically increased tumor control, both in terms of tumor burden and overall survival, when compared to Mock KO CART5 (median overall survival Mock KO CART5=62 days vs. CD5 KO CART5=undefined; p=0.0062, Mantel-Cox) (FIG. 6B-6C). This enhanced anti-tumor effect is likely associated with the increased expansion of CD5 KO CART5 cells in the peripheral blood (PB) (454.00±118.50 cells/100 L PB vs. 19.54±6.84 cells/100 L PB in Mock KO CART5; p=0.0064) (FIG. 6D).

Upon in vitro validation, NSG mice were engrafted with mesothelin+ AsPC1 pancreatic cancer cells and subsequently treated with CARTmeso or untransduced controls. CD5 KO CARTmeso cells demonstrated strong anti-tumor activity as compared to controls as validated through both tumor volume and bioluminescence measurements (FIG. 2G). CD5 KO CARTmeso cells displayed increased numbers of T cells in the peripheral blood (FIG. 2G). To test whether CD5 KO CARTmeso cells are able to establish prolonged immune memory, long-term surviving mice were rechallenged with the same tumor. CD5 KO CARTmeso cells did not show tumor engraftment while control mice did.

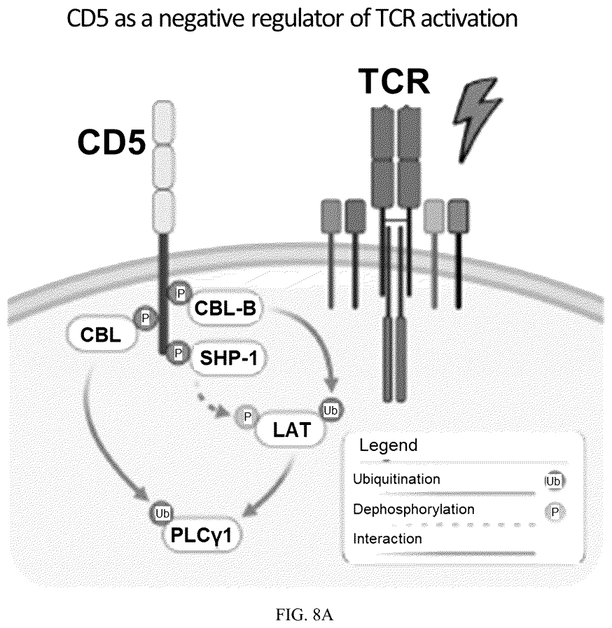

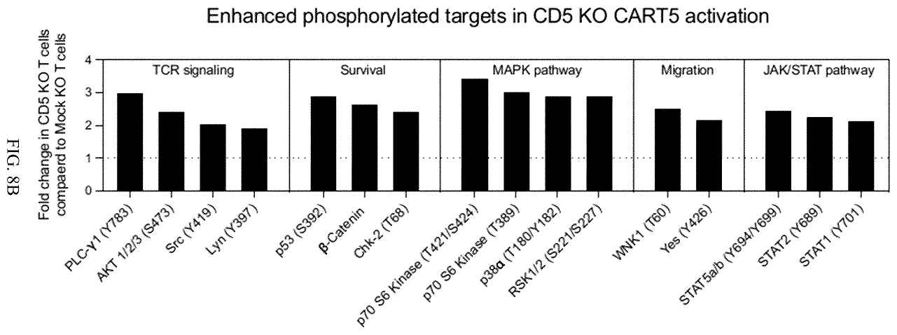

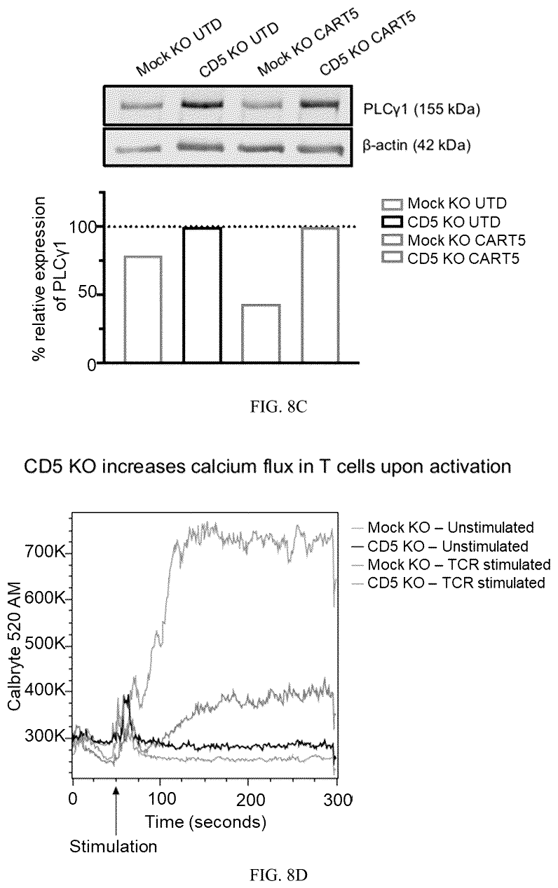

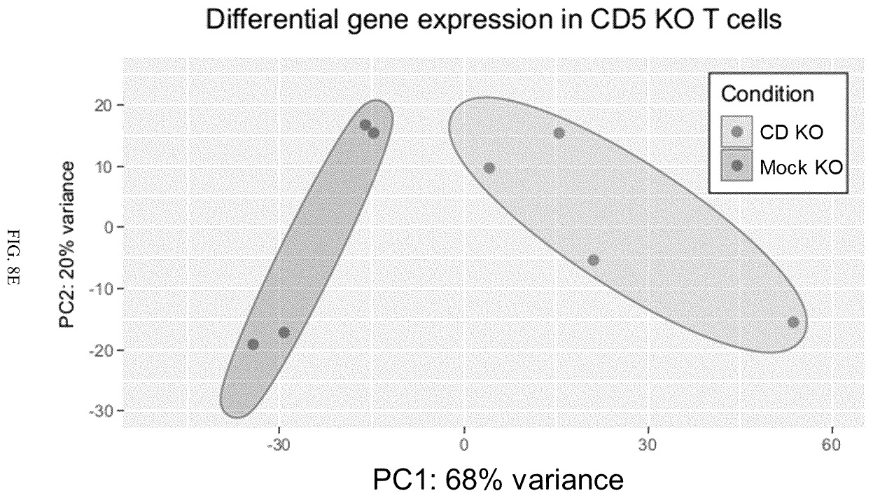

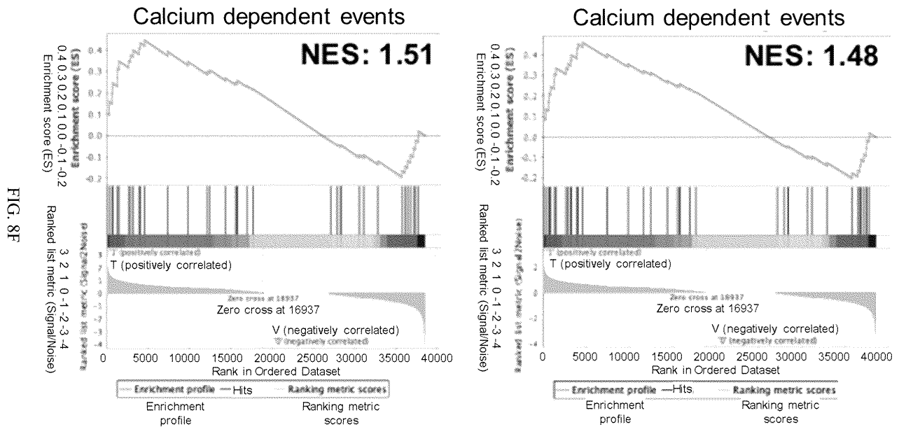

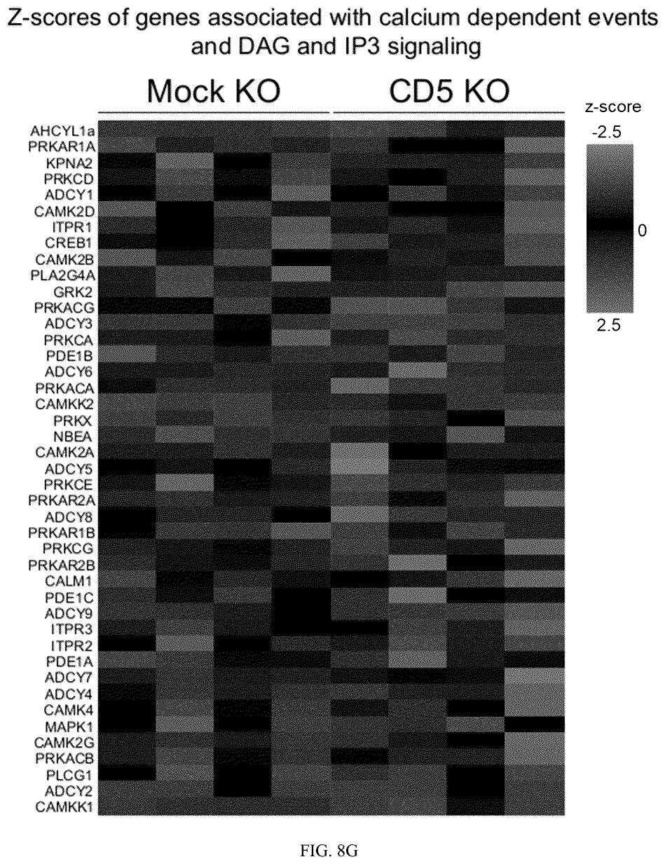

CD5 acted as a negative regulator of T cell activation in CAR T cells: Given the high killing activity of CD5 KO CART5 cells in vivo, the next aim was to define the mechanisms by which CD5 KO enhances CAR T cell anti-tumor efficacy. In the context of TCR activation, CD5 recruited several inhibitory mediators to the cell membrane such as SHP-1, CBL, and CBL-B (FIG. 8A). These factors are known to negatively regulate T cell activation by diminishing PLC7 activity, a key kinase involved in activating transcription factors AP-1 and NF-κB as well as intracellular calcium release to activate transcription factor NFAT. From this, the hypothesis was that the deletion of CD5 may abrogate these mediators' functions, leading to increased PLC7 signaling and enhanced CAR activity. To test this hypothesis, an unbiased phospho-kinase array was used to simultaneously detect the relative levels of phosphorylation of 37 kinase phosphorylation sites 15 minutes after CART5 stimulation. Remarkably, CD5 KO CART5 cells showed higher (>2 fold) phosphorylation of several signaling proteins, including key regulators of T cell activation (PLC71, AKT) and proliferation (p70 S6, β-Catenin) when compared to Mock KO CART5 (FIG. 8A). The total PLC7 expression was observed to have higher levels in CD5 KO cells compared to Mock KO T cells (FIG. 8C). CD5 KO cells also presented drastically higher levels of calcium than Mock KO cells (FIG. 8D). Bulk RNA sequencing analysis of unstimulated Mock KO and CD5 KO cells demonstrated strong differential gene expression between the two groups (FIG. 8E). Interestingly, gene set enrichment analysis revealed several pathways related to the PLC7 pathway such as calcium-dependent events and DAG-IP3 signaling (FIG. 8F-8G) to be enriched in CD5 KO cells. These data further suggested that CD5 can act as an inhibitory molecule in T cells and that its KO could prime CAR T cells to be more prone to increased activation upon stimulation.

Depletion of CD5 enhanced solid tumor targeting CARTmeso cells: To test the hypothesis that deletion of CD5 could increase the anti-tumor effect of CAR T cells targeting antigens other than CD5, CD5 was knocked out in CD5 KO CAR T cells in a solid cancer model of PDAC. NSG mice were engrafted with mesothelin+ AsPC1 and subsequently treated with anti-mesothelin CAR (CARTmeso) T cells. When compared to Mock KO CARTmeso, CD5 KO CARTmeso cells demonstrated strong tumor control as validated through both tumor volume (FIG. 9A) as well as tumor burden via bioluminescence (FIG. 9B). CD5 KO CAR T cells once again displayed increased numbers of T cells in the peripheral blood (7.71×10⁵±3.02×10⁵ cells/100 μL PB vs. 2.27×10⁵±2.21×10⁵ cells/100 L PB in Mock KO CARTmeso; p=ns). Notably, the number of T cells detected in the peripheral blood for all groups was considerably higher in this solid tumor model compared to previous liquid tumor models at the same timepoint. Despite the strong tumor control achieved by CD5 KO CARTmeso cells, this group died at a more rapid pace than the Mock KO CARTmeso group, likely due to the increased T cell count contributing to graft-versus-host disease and not the tumor itself. In another experiment of the same model using a challenging low-dose of CARTmeso cells (0.2×10⁶ CAR+ cells injected on Day 0 versus 0.75×10⁶ CAR+ cells injected in initial experiment), CD5 KO CARTmeso cells demonstrated efficient tumor control further correlating with increased survival (median overall survival Mock KO CARTmeso=42 days vs. CD5 KO CARTmeso=102 days; p=0.14, Mantel-Cox). This enhanced survival is again likely associated to the increased expansion and persistence of T cells (FIG. 9F) that were able to further control re-challenge of AsPC1 tumor cells injected on Day 74 (FIG. 9G).

To answer the question of whether the expression of CD5 on tumor-infiltrating lymphocytes in biopsies of cancer patients impacted overall survival, the TCGA database was analyzed and it was identified that low CD5 expression (normalized by CD3F) in pretreatment biopsies correlated with significantly improved overall survival in over 9,000 cancer patients (FIG. 9H). These results were particularly remarkable in 459 patients with the highly immunogenic skin cutaneous melanoma (FIG. 9I).

In conclusion, this study demonstrated for the first time that CD5 is a negative regulator and possible novel immune checkpoint for CAR T cell immunotherapy. It was shown that CD5 deletion led to enhancement of CAR T function in vivo in several clinically relevant models of liquid and solid tumors. These findings support the development of CD5 KO CAR T products in early-phase clinical trials.

### Example 3

Anti-HER2 CAR T cells (Clone 4D5, J Immunol. 2009 Nov. 1; 183(9): 5563-5574, which is hereby incorporated by reference in its entirety) were generated following the manufacturing timeline previously described. As a model to test the efficacy of CD5 KO anti-HER2 CAR T cells, PC3, a HER2+ prostate adenocarcinoma was used. 4,000 PC3 cells were plated in a 96-well plate 24 hours prior to addition of 1000 HER2+ CAR T cells or controls. GFP+(PC3) intensity was monitored every 3 hours using the Incucyte® Live-Cell Analysis System (FIG. 10A). Significantly higher cytotoxicity was demonstrated with the CD5 KO compared to the wild type anti-HER2 CAR (FIG. 10B).

### Discussion

CAR T cell therapy has generated considerable enthusiasm in the treatment of B cell leukemias and lymphomas. Currently, all but one of the FDA-approved CAR T cell therapies target the B cell surface protein, CD19. While this has emerged as a standard approach for patients with relapsed and refractory B cell disease, there is still a significant fraction of patients who eventually relapse. In addition, many challenges arise when translating this therapy for other malignancies such as T cell related cancers or solid tumors. Thus, there is a need in the art to develop effective strategies to overcome these issues.

T cells modified by lentiviral insertion of a CAR transgene to redirect their immune specificity greatly increased the anti-tumor immune response of a patient. Combining this method with CRISPR-Cas9 to further modify T cells presented a method to improve the function of CAR T cells. Several groups have reported improved anti-tumor activity of CAR T by CRISPR-Cas9 gene editing of different genes, or used it to silence TCR locus genes to generate off the shelf CAR T cells. The usage of CRISPR-Cas9 technique has been demonstrated to be safe for patients. In theory, CRISPR-Cas9 can be used to ameliorate the CAR T therapy and expand the possible targets.

Here, it was established that CRISPR-Cas9 KO of CD5 increased the anti-tumor efficacy of several different antigen and tumor models of CAR T cells. In the context of CD5 targeting CAR T cells in T cell malignancies, the genetic deletion of CD5 eliminated potential fratricidal concerns which ultimately led to significantly increased levels of desired memory phenotypes and reduced exhaustion markers. These characteristics of CD5 KO CART5 cells allowed for a more functional product which outperformed Mock KO CART5 cells and greatly extended the life of mice in vivo. This increased activity can likely be contributed to increased expansion of CAR T cells at later timepoints. To better understand the potential mechanisms behind this increased T cell proliferation, unbiased proteomic and genomic approaches were used to identify PLC71 as an important mediator. Upon T cell activation, CD5 is known to recruit several proteins to its cytoplasmic tail including SHP-1, CBL, and CBL-B. These proteins, by various pathways, are known to act as negative regulators of T cell signaling, especially through diminishing PLC71 activity. From this, the hypothesis was that depletion of CD5 in CAR T cells can lead to these mediators being unable to perform their inhibitory functions, thereby increasing proliferation.

To better understand whether the enhanced effects of CD5 KO in CART5 are solely due to eliminating fratricidal concerns, or may in part be related to preventing the TCR signaling inhibitory effects of CD5, CD5 was deleted in additional antigen and tumor models of CAR T cells. CD5 KO was tested in mesothelin-targeting CAR T cells against pancreatic ductal adenocarcinoma, an example of a solid tumor model that CAR T therapy. Interestingly, the deletion of CD5 greatly heightened the anti-tumor abilities of CAR T cells, suggesting that its role as a negative regulator of T cell signaling may have significant effects for adoptive T cell therapy.

The generation of CAR T product effective against T cell lymphomas is complicated due to the fact that most of the targetable antigen expressed on the surface of neoplastic cells are shared with the normal T cells counterpart used for CAR T production, leading, thus, to fratricide killing and exhausted product. Different CARTs have been investigated in T-ALL and in T cell lymphoma resulting in controversial results. The CD5 KO CART5 presented herein avoids these issues. Higher anti-neoplastic killing activity was demonstrated in a CD5 KO animal model that displayed a less exhausted phenotype.

The hypothesis was that CRISPR-Cas9 deletion of the antigen targeted by CAR is an optimal strategy to reduce fratricide and exhausting interactions to produce new effective therapy in T cell neoplasms. This hypothesis was tested herein. These preclinical observations demonstrated that CD5 KO CART5 is an interesting product that needs to be investigated in clinical studies to confirm its beneficial role and anti-neoplastic role.

### Methods

Cell lines and primary samples: Unless otherwise specified, all cell lines were cultured in Roswell Park Memorial Institute medium 1640 (RPMI; Gibco; Cat #11875-085) supplemented with 10% fetal bovine serum (FBS, Gibco; Cat #16140-071), 1% penicillin and 1% streptomycin (Gibco; Cat #15140-163), 1% GlutaMAX supplement (Gibco; Cat #35050-079, and 1% HEPES (Gibco; Cat #15630-130) in a 37° C. incubator with 5% CO₂. All cell lines were authenticated by short tandem repeat (STR) analysis and tested for mycoplasma using a MycoAlert Plus Mycoplasma Detection Kit (Lonza; Cat #LT07-710). Nalm6, Jurkat, HEK293T, and AsPC1 cell lines were purchased from American Type Culture Collection (ATCC).

CD5 sgRNA optimization: CRISPR sgRNAs were designed using software integrated into Benchling (https://www.benchling.com). For each target gene, eight sgRNA sequences were designed to target early exon sequences, and in vitro transcribed using the GeneArt Precision gRNA Synthesis Kit (Invitrogen; Cat #A29377) for screening. Cells were electroporated using the Lonza 4D-Nucleofector Core Unit. Primary T cells were electroporated using the P3 Primary Cell 4D-Nucleofector X Kit L (Lonza; Cat #V4XP-3024). For Cas9 and sgRNA delivery, the ribonucleoprotein (RNP) complex was initially formed by incubating 10 g of TrueCut Cas9 Protein v2 (Lonza; Cat #A36499) with 5 g of sgRNA for 10 minutes at room temperature for every. 10×10⁶ cells were spun down at 300× g for 5 minutes and resuspended in 100 L in the specified buffer. The RNP complex and 100 μL of resuspended cells were combined and electroporated using pulse code EO-115. After electroporation, the cells were incubated in standard media containing 20 ng/mL of supplemental cytokines TL-7 and IL-15 at a concentration of 2×10⁶ cells/mL at 37° C. CD5 expression was subsequently monitored at each of the indicated days.

Anti-CD5 CAR single-chain fragment variable design: CARs targeting CD5 were generated based on the antibody variable region sequences of monoclonal antibodies 9, 17, and 34. Single chain variable fragments (scFv) were designed in both orientations (from variable light chain to variable heavy chain and vice versa) with three glycine-serine-serine-serine linkers and synthesized at GenScript Biotech. After initial CART5 screening, all experiments were performed using clone #17 in a heavy-to-light chain orientation (17H2L).

Lentiviral vector production: Replication-defective, third-generation lentiviral vectors were produced using HEK293T cells. Approximately 8×10⁶ cells were plated in T150 culture vessels in standard culture media and incubated overnight at 37° C. 18-24 h later, cells were transfected using a combination of Lipofectamine 2000 (116 L; Invitrogen; Cat #11668-019), pVSV/G (7 g), pRSV/Rev (18 g), pGag/Pol (18 g) packaging plasmids and 15 g of expression plasmid (CART5, or CARTmeso). Lipofectamine and plasmid DNA were diluted in 4 mL Opti-MEM media (Gibco; Cat #31985-070) before transfer into lentiviral production flasks. At both 24 and 48 h following transfection, culture media was isolated and concentrated using high-speed ultracentrifugation (25,000×g for 2.5 hours).

Manufacturing of primary human genome-engineered CAR T cells: Human T cells were procured through the University of Pennsylvania Human Immunology Core. CD4+ and CD8+ cells were combined at a 1:1 ratio and used for electroporation. CRISPR-Cas9 sgRNAs targeting CD5 was chemically synthesized (Integrated DNA Technologies) and 5 g sgRNA were premixed with 10 g of TrueCut Cas9 Protein v2 (Invitrogen; Cat #A36499) for 10 minutes at room temperature to form a ribonucleoprotein (RNP) complex prior to electroporation. 10×10⁶ T cells in 100 L of the buffer provided with P3 Primary Cell 4D-Nucleofector X Kit L (Lonza; Cat #V4XP-3024) were mixed with the RNP complex and subsequently electroporated using the pulse code EO-115 in a 4D-Nucleofector (Lonza; Cat #AAF-1002B). Mock KO cells were electroporated using the same procedure as described without the presence of an RNP complex. After electroporation, T cells were incubated at 37° C. for 24 hours and subsequently activated using CD3/CD28 Dynabeads (Gibco; Cat #40203D) at a ratio of 3 beads/cell. The following day, CAR lentiviral vectors were added to stimulated cultures at a multiplicity of infection between 1 and 3. Beads were removed between days 6-8 of stimulation, and cells were counted every other day using a Multisizer 3 Coulter Counter (Beckman) until growth kinetics and cell size demonstrated they had rested from stimulation. All T cells were initially grown with 20 ng/mL of supplemental cytokines IL-7 and IL-15 that was decreased to 0 ng/mL by the end of the expansion. All CAR constructs were composed of an scFV (CAR5, 17H2L; FMC63; CARmeso, CARM5), 4-1BB co-stimulatory domain, and CD3C co-stimulatory domain, unless otherwise noted.

General flow cytometry: Cells were resuspended in FACS staining buffer (phosphate buffered saline (PBS)+2% FBS) using one or more of the following antibodies: CD5 (UCHT2; BioLegend; Cat #300612), CD4, CD8 (RPA-T8; BD Biosciences; Cat #563795), CD45RA (L48; BD Biosciences; Cat #347723), CCR7 (G043H7; BioLegend; Cat #353214), PD1 (EH12.1; BD Biosciences; Cat #562516), LAG3 (3DS223H; Invitrogen; Cat #11-2239-42), CD3 (UCHT1; BD Biosciences; Cat #565119 or OKT3; BioLegend; Cat #317334), hCD45 (2D1; BioLegend; Cat #368522), or mCD45. CAR5 was detected by incubating cells with recombinant human CD5 protein conjugated to a polyhistidine (His) tag at the C-terminus (Sino Biologicals; Cat #11027-H08H) followed by an Allophycocyanin (APC) anti-6× His-tag secondary antibody (Abcam; Cat #ab72579). CARmeso was detected using a Biotin-SP goat anti-human F(ab′)₂ antibody (Jackson ImmunoResearch; Cat #109-066-097) followed by an R-Phycoerythrin (PE) streptavidin secondary antibody (Jackson ImmunoResearch; Cat #016-110-084). All changes in overall tumor or T cell counts reflected by absolute cell counts were determined using Flow-Count Fluorospheres (Beckman; Cat #754053). Cell viability was established using ViaKrome 808 Fixable Viability Dye (Beckman; Cat #C36628) and data were acquired on an LSRII Fortessa Cytometer (BD) or CytoFlex LX Flow Cytometer (Beckman). All data analysis was performed using FlowJo 10.8.0 software (FlowJo, LLC).

iGenome-wide, Unbiased Identifications of DSB Enabled by Sequencing (iGUIDE-Seq): 10×10⁶ primary T cells were electroporated without or with 10 g TrueCut Cas9 Protein v2 (Invitrogen; Cat #A36499) or SpyFi™ Cas9 Nuclease (Aldevron; Cat #9214-0.25MG) and 5 g of CD5 targeting sgRNA in the presence of double-stranded oligodeoxynucleotides (dsODNs, a unit of measure associated with iGUIDE or GUIDE-seq based analyses), stimulated at a ratio of 3 beads/cell, and transduced with CART5. Cells were debeaded and collected on days 3 or 5 and genomic DNA was isolated using DNeasy Blood & Tissue Kit (Qiagen; Cat #69504). iGUIDE-Seq and analysis was employed on the isolated DNA to detect and quantify on- and off-target cleavage sites and associated deletions.

CART5 on- and off-targeting specificity analysis: For a library screen, CD5 KO CART5 cells were fluorescently labelled and screened for binding against fixed HEK293 cells/slides expressing 5828 human plasma membrane proteins and cell surface tethered human secreted proteins+398 human heterodimers in duplicates (18 slide sets, n=2 slides per slide set, 3.2×10⁷ cells/slide). In total, 22 primary hits (duplicate spots) were identified by analyzing fluorescence (Alexa Fluor 647 and ZsGreen1) on ImageQuant. There were a range of intensities (signal to background) from very weak to strong. A confirmation screen was performed by testing Mock KO UTD and CD5 KO CART5 cell binding against all 22 hits in duplicate on new slides. Library and confirmation screens as well as analyses were performed at Retrogenix, Charles River Laboratories.

Bioluminescence-based cellsurvival assays: Cell lines (Jurkat, Nalm6, AsPC1, OCI-Ly18, SU-DHL-4, and SU-DHL-2) were engineered to express click beetle green (CBG), and cell survival was measured using bioluminescent quantification. D-luciferin potassium salt (Gold Biotechnology; Cat #115144-35-9) was added to cell cultures (final concentration 15 g/mL) and incubated at 37° C. for 10 min. Bioluminescent signal was detected using a BioTek Synergy H4 imager, and signal was analyzed using BioTek Gen5 software. Percent cytotoxicity was calculated using a control of target cells without effectors.

Flow cytometry-based cell survival assays: Primary T-ALL or Sezary cells were stained with Cell Trace Violet (CTV; Invitrogen) prior to plating with CAR T cells or control untransduced T cells. Reagents were used according to manufacturer protocol. After 48 h, cells were stained with ViaKrome 808 Fixable Viability Dye (Beckman; Cat #C36628) and analyzed by flow cytometry to determine absolute count Flow-Count Fluorospheres (Beckman; Cat #754053) of ViaKrome 808-negative and CTV-positive cells. Absolute cell counts were determined using Flow-Count Fluorospheres (Beckman; Cat #754053). Percent cytotoxicity was calculated using a control of target cells without effectors.

Xenograft mouse models: 6-10-week-old NOD-SCID-γc^{-/-} (NSG) mice were obtained from the Jackson Laboratory and maintained in pathogen-free conditions. All target AsPC1 cells were engineered to express click beetle green (CBG). For solid tumor experiments, animals were injected subcutaneously with 2×10⁶ AsPC1 cancer cells as indicated in 0.2 mL Matrigel. 23-27 days after tumor delivery, 0.75×10⁶ (high-dose) or 0.2×10⁶ (low-dose) T cells (control or CAR+) were injected via tail vein in 0.15 mL sterile PBS. Animals were monitored for signs of disease progression and overt toxicity, such as xenogeneic graft-versus-host disease, as evidenced by >10% loss in body weight, loss of fur, diarrhea, conjunctivitis and disease-related hind limb paralysis. Disease burdens were monitored over time using a Xenogen IVIS bioluminescent imaging system or the Lumina S5 imaging system.

Kinase phosphorylation profiling: Phosphorylated proteins were quantified using the Human Phospho-Kinase Array Kit (R&D Systems). To activate cells through their CAR, CD5-coated magnetic beads were prepared using recombinant CD5 protein (Sino Biological; Cat #11027-H08H) and Dynabeads M450 tosylactivated (Invitrogen; Cat #14013) according to the manufacturer protocol. 10×10⁶ T cells were mixed with CD5-conjugated beads at a ratio of 4 beads per T cell and incubated at 37° C. for 30 minutes. Cells were then lysed according to the array kit protocol, and phospho-peptides were detected and analyzed using quantitative chemiluminescence. Images were captured using ImageQuant LAS 4010 (GE Healthcare) and densitometry analysis was performed with Image Studio Lite Version 5.2 (LI-COR Biotechnology). The amount of each target protein molecules was normalized with internal reference values (pixel density of reference spots). Relative phospho-peptide quantities were determined by comparing normalized signal intensity in CD5 KO CART5 to Mock KO CART5.

Immunoblot: Cell lysates were prepared in IP lysis buffer (Pierce; Cat #87787) with Halt protease and phosphatase inhibitor cocktail (Thermo Fisher; Cat #1861281) according to the manufacturer protocol. Protein concentrations were measured using Rapid Gold BCA Protein Assay Kit (Pierce; Cat #A53226). Protein samples were reduced in 4× protein sample loading buffer (LI-COR; Cat #928-40004) with 5% β-mercaptoethanol (Gibco; Cat #21985-023) and boiled at 95° C. for 10 minutes (or 70° C. for 10 minutes for membrane protein). Denatured protein samples were then loaded onto 4-15% SDS protein gel (Bio-Rad; Cat #4561083). After polyacrylamide gel electrophoresis, proteins were transferred to PVDF membranes for standard immunoblotting. Followed by blocking step, protein targets were detected with the following primary antibodies that were purchased from Cell Signaling Technology: Total PLCγ1 (D9H10; Cat #5690S), β-actin (8H10D10; Cat #3700S), and CD5 (E8X3S; Cat #39300S). Fluorescence dye conjugated secondary antibodies (LI-COR; Cat #926-68072 and 925-32213) were used for detecting primary antibodies. Fluorescence signals were detected with Odyssey CLx imaging system (LI-COR) and densitometry analysis was performed with Image Studio Lite Version 5.2 (LI-COR Biotechnology).

Flow cytometry-based calcium flux assay: Calcium influx in T cells were measured using Calcium Flux Assay Kit (abcam; Cat #ab233472) according to the manufacturer protocol. Briefly, 0.5×10⁶ cells were incubated with 520 AM dye-loading solution at 37° C. for 30 minutes. Cells were then centrifuged at 300×g for 5 minutes and buffer was replaced with Hank's Balanced Salt Solution with 20 mM HEPES (HHBS) solution. For activation, cells were incubated with anti-CD3 antibody (OKT3, BioLegend, Cat #317302) at a concentration of 10 g/mL for 15 minutes on ice, followed by goat anti-mouse IgG secondary antibody (Invitrogen; Cat #A32728) incubation at concentration of 10 g/mL. For data acquisition, baseline signals were recorded for 60 seconds, and activation signals were recorded for 5 minutes right after mixing secondary antibody solution. Signal of fluorescence was read using a CytoFlex LX Flow Cytometer (Beckman Coulter) and kinetics of median fluorescence events with gaussian smoothing algorithm were plotted using FlowJo 10.8.0 software (FlowJo, LLC).

RNA sequencing and analysis: RNA sequencing was performed by the Perelman School of Medicine Next-Generation Sequencing Core (Philadelphia, PA) on a NovaSeq 6000 Sequencing System (Illumina) as 100 base paired-end reads using an NEBNext Ultra II Directional RNA Library Prep Kit for Illumina (New England BioLabs; Cat #E7760L). Reads of the samples were trimmed for adapters and low-quality bases using Trimmomatic (version 0.36) before alignment with the hg19 reference genome and the annotated transcripts using Spliced Transcripts Alignment to a Reference (STAR; version 2.6.0c). Gene expression quantification analysis was performed for all samples using STAR and featureCounts (version 16.1). Differential expression analyses between samples were computed using DESeq2 (version 1.22.2)²⁴ within R Studio (version 1.2.5042). Pathway enrichment was performed using the Gene Set Enrichment Analysis (GSEA) Mac App (version 4.1.0) with the Hallmark gene set from the Molecular Signatures Database.

TCGA dataset analysis: The GEPIA2 TCGA database was used to investigate the role of CD5 expression in pretreatment biopsies in 9502 patients. CD5 expression was normalized to CD3F to account for tumor-infiltrating lymphocyte T cell numbers. Patients were divided in two groups based on high and low CD5 expression (75% high; 25% low).

Statistical analysis: Data were visualized and analyzed using Prism 9 software (GraphPad). All results are represented as either individual values or as mean values ±standard error of the mean (SEM). All comparisons between two groups were performed using two-tailed unpaired Student's t-test. Comparisons between more than two groups were performed by one-way analysis of variance (ANOVA) with Tukey correction for multiple comparisons. Survival data were analyzed using the Log-Rank (Mantel-Cox) test. The p values were denoted with asterisks as follows: * p<0.05, ** p<0.01, *** p<0.001, **** p <0.0001.

TABLE 1

| Name | Sequence | SEQ ID NO |
|---|---|---|
| CD5_gRNA_1 | CTCTCTGAGAGCGAGATACC | 70 |
| CD5_gRNA_2 | CGGGTATCTCGCTCTCAGAG | 71 |
| CD5_gRNA_3 | CATACCAGCTGAGCCGTCCG | 72 |
| CD5_gRNA_4 | CGGCTCAGCTGGTATGACCC | 73 |
| CD5_gRNA_5 | GCAGACTTTTGACGCTTGAC | 74 |
| CD5_gRNA_6 | AATCATCTGCTACGGACAAC | 75 |
| CD5_gRNA_7 | GGCGGGGGCCTTGTCGTTGG | 76 |
| CD5_gRNA_8 | AGACAACACCTCCAACGACA | 77 |

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While the embodiments provided for herein have been disclosed with reference to specific embodiments, it is apparent that other embodiments and variations of these may be devised by others skilled in the art without departing from the true spirit and scope of the present disclosure. The appended claims are intended to be construed to include all such embodiments and equivalent variations.

### Enumerated Embodiments

The following enumerated embodiments are provided, the numbering of which is not to be construed as designating levels of importance.

Embodiment 1 provides a method of treating a solid tumor in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen.

Embodiment 2 provides a method of treating a solid tumor in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, wherein the growth of the tumor is inhibited for at least 10, 20, 30, 40, 50, 60, 70, 80, 90, 100, 120, 150, 200, 240 days.

Embodiment 3 provides a method of treating a solid tumor with enhanced efficacy in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, and wherein the efficacy of the population of immune cells comprising the mutated endogenous CD5 gene is greater than a population of immune cells comprising a non-mutated endogenous CD5 gene.

Embodiment 4 comprises the method of any of the preceding embodiments, wherein the pharmaceutical composition comprises about 1×10⁶ to about 1×10⁹, about 1×10⁷ to about 1×10⁹, about 1×10⁸ to about 1×10⁹, about 2×10⁸ to about 1×10⁹, about 3×10⁸ to about 1×10⁹, about 3×10⁸ to about 1×10⁹, about 3×10⁸ to about 1×10⁹, about 4×10⁸ to about 1×10⁹, about 5×10⁸ to about 1×10⁹, about 6×10⁸, about 7×10⁸ to about 1×10⁹, about 8×10⁸, about 9×10⁸ to about 1×10⁹ of the immune cells.

Embodiment 5 comprises the method of any of the preceding embodiments, wherein at least 75%, 80%, 85%, 90%, 91%, 92%, 93%, 94%, 95%, 96%, 97%, 98%, 99%, or 100% of the population of immune cells comprise the mutated endogenous CD5 gene.

Embodiment 6 comprises the method of any of the preceding embodiments, wherein at least 50%, 55%, 60%, 65%, 75%, 80%, 85%, 90%, 91%, 92%, 93%, 94%, 95%, 96%, 97%, 98%, 99%, or 100% of the population of immune cells comprise the heterologous chimeric antigen receptor (CAR).

Embodiment 7 provides the method of any of the preceding embodiments, wherein the immune cells do not express or comprise an endogenous full length CD5 protein.

Embodiment 8 provides the method of any of the preceding embodiments, wherein the mutated CD5 gene has an exon 1 or exon 2 mutation, insertion or deletion.

Embodiment 9 provides the method of embodiment 8, wherein the exon 1 or exon 2 mutation, insertion or deletion inhibits or reduces the expression of the endogenous full length CD5 protein.

Embodiment 10 provides the method of any of the preceding embodiments, wherein the endogenous CD5 gene comprises a gene edited mutation, insertion or deletion.

Embodiment 11 provides the method of embodiment 10, wherein the gene edited mutation, insertion or deletion is a CRISPR/Cas9 mediated gene mutation, insertion or deletion.

Embodiment 12 provides the method of embodiment 11, wherein the CRISPR/Cas9 mediated gene mutation, insertion or deletion targets exon 1 or exon 2 of the CD5 gene.

Embodiment 13 provides the method of any of the preceding embodiments, wherein the gene edited or CRISPR/Cas9 mediated gene mutation, insertion or deletion utilizes an sgRNA comprising the nucleotide sequence of SEQ ID NO: 45.

Embodiment 14 provides the method of any preceding embodiment, wherein the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), 0-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Embodiment 15 provides the method of any preceding embodiment, wherein the modified immune cell is a T cell.

Embodiment 16 provides the method of any preceding embodiment, wherein the antigen binding domain of the CAR comprises a complementarity determining region (CDR) comprising an amino acid sequence selected from the group consisting of SEQ ID NOs: 27-32 and 39-44.

Embodiment 17 provides the method of any preceding embodiment, wherein the antigen binding domain of the CAR comprises a heavy chain variable region comprising the amino acid sequence selected from the group consisting of SEQ ID NOs: 19, 25, and 37and/or a light chain variable region comprising the amino acid sequence selected from the group consisting of SEQ ID NOs: 20, 26, and 38.

Embodiment 18 provides the method of any preceding embodiment, wherein the antigen binding domain of the CAR comprises an scFv comprising the amino acid sequence selected from the group consisting of SEQ ID NOs: 17, 18, 23, 24, 35, or 36.

Embodiment 19 provides the method of any preceding embodiment, wherein the CAR comprises an amino acid sequence selected from the group consisting of SEQ ID NOs: 15, 16, 21, 22, 33, and 34.

Embodiment 20 provides the method of any preceding embodiment, wherein the CAR is encoded by a nucleic acid sequence selected from the group consisting of SEQ ID NOs: 1-6.

Embodiment 21 provides the method of any preceding embodiment, wherein the solid tumor is prostate cancer (e.g., pancreatic ductal adenocarcinoma (“PDAC”), fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

Embodiment 22 provides a method of treating a solid tumor in a subject in need thereof, the method comprising: administering to the subject a vector comprising a targeting moiety that binds to a CD5 expressing immune cell and a polynucleotide encoding a gene editing system and a chimeric antigen receptor (CAR), wherein the CAR comprises an antigen binding domain, a transmembrane domain, and an intracellular domain, wherein the antigen binding domain binds to a solid tumor antigen, wherein the gene editing system is configured to modify the endogenous CD5 gene to inhibit, eliminate, or reduce expression of endogenous CD5, and wherein the vector transduces a CD5 expressing immune cell in the subject to mutate the endogenous CD5 gene to inhibit or reduce expression of endogenous CD5 and express the CAR in the immune cell with the mutated endogenous CD5 gene.

Embodiment 23 provides the method of embodiment 22, wherein the vector is a viral vector.

Embodiment 24 provides the method of embodiment 23, wherein the viral vector is a lentivirus.

Embodiment 25 provides the method of any one of embodiments 22-24, wherein the targeting moiety is an antibody or other type of targeting moiety (e.g. a scFv, an antigen binding domain, a DARPIN, a VHH, or a FN3 domain).

Embodiment 26 provides the method of any one of embodiments 22-25, wherein the solid tumor is fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

Embodiment 27 provides the method of any one of embodiments 22-26, wherein the transduced cells do not express or comprise an endogenous full length CD5 protein.

Embodiment 28 provides the method of any one of embodiments 22-27, wherein the mutated CD5 gene has an exon 1 or exon 2 mutation, insertion or deletion.

Embodiment 29 provides the method of claim 28, wherein the exon 1 or exon 2 mutation, insertion or deletion inhibits or reduces the expression of the endogenous full length CD5 protein.

Embodiment 30 provides the method of any one of embodiments 22-29, wherein the endogenous CD5 gene comprises a gene edited (e.g., CRISPR mediated) gene mutation, insertion or deletion.

Embodiment 31 provides the method of embodiment 30, wherein the gene edited gene mutation, insertion or deletion is a CRISPR/Cas9 mediated gene mutation, insertion or deletion.

Embodiment 32 provides the method of embodiment 30 or 31, wherein the gene edited gene mutation, insertion or deletion targets exon 1 or exon 2 of the CD5 gene.

Embodiment 33 provides the method of embodiment 32, wherein the gene edited (e.g., CRISPR/Cas9 mediated) gene mutation, insertion or deletion utilizes an sgRNA comprising the nucleotide sequence of SEQ ID NO: 45.

Embodiment 34 provides the method of any one of embodiments 22-33, wherein the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), 0-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Embodiment 35 provides the method of any preceding embodiment, wherein the modified immune cell is a T cell.

Embodiment 36 provides the method of any preceding embodiment, wherein the antigen binding domain of the CAR comprises a complementarity determining region (CDR) comprising an amino acid sequence selected from the group consisting of SEQ ID NOs: 27-32 and 39-44.

Embodiment 37 provides the method of any preceding embodiment, wherein the antigen binding domain of the CAR comprises a heavy chain variable region comprising the amino acid sequence selected from the group consisting of SEQ ID NOs: 19, 25, and 37 and/or a light chain variable region comprising the amino acid sequence selected from the group consisting of SEQ ID NOs: 20, 26, and 38.

Embodiment 38 provides the method of any preceding embodiment, wherein the antigen binding domain of the CAR comprises an scFv comprising the amino acid sequence selected from the group consisting of SEQ ID NOs: 17, 18, 23, 24, 35, or 36.

Embodiment 39 provides the method of any preceding embodiment, wherein the CAR comprises an amino acid sequence selected from the group consisting of SEQ ID NOs: 15, 16, 21, 22, 33, and 34.

Embodiment 40 provides the method of any preceding embodiment, wherein the CAR is encoded by a nucleic acid sequence selected from the group consisting of SEQ ID NOs: 1-6.

Embodiment 41 provides a modified immune cell comprising a chimeric antigen receptor (CAR), wherein the CAR comprises an antigen binding domain, a transmembrane domain, and an intracellular domain, and wherein the endogenous CD5 gene has been mutated, modified, disrupted, or knocked-out, and wherein the antigen binding domain binds to a solid tumor antigen.

Embodiment 42 provides the modified immune cell of claim 36, wherein the antigen binding domain of the CAR binds to mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, pi85erbB2, pi80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Embodiment 43 provides a composition (e.g., a pharmaceutical composition) comprising population of the modified immune cells of embodiment 41 or 42.

Embodiment 44 provides the composition of embodiment 43, wherein at least 75%, 80%, 85%, 90%, 91%, 92%, 93%, 94%, 95%, 96%, 97%, 98%, 99%, or 100% of the population of the modified immune cells comprise the mutated endogenous CD5 gene.

Embodiment 45 provides the composition of embodiment 43 or 44, wherein at least 50%, 55%, 60%, 65%, 75%, 80%, 85%, 90%, 91%, 92%, 93%, 94%, 95%, 96%, 97%, 98%, 99%, or 100% the population of immune cells comprise the heterologous chimeric antigen receptor (CAR).

Embodiment 46 provides a method of generating the modified immune cell of any of the preceding embodiments, the method comprising transfecting or transducing the immune cell with a nucleic acid encoding the CAR and/or at least one nucleic acid that knocks-out, mutates, or disrupts endogenous CD5 gene, such as an exon of the CD5 gene, including but not limited to exon 1 or exon 2 of the CD5 gene, wherein the CAR comprises an antigen binding domain, a transmembrane domain, and an intracellular domain, and wherein the antigen binding domain binds to a solid tumor antigen.

Embodiment 47 provides the method of embodiment 46, wherein the antigen binding domain binds to mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, pi85erbB2, pi80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Embodiment 48 provides the method of embodiment 46 or 47, wherein the immune cell is transduced in vivo or ex vivo.

Embodiment 49 provides the method of claim 48, wherein the immune cell is transduced in vivo.

Embodiment 50 provides the method of any of the preceding embodiments, wherein the immune cell is a T cell.

1. A method of treating a solid tumor in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen.

2. A method of treating a solid tumor in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, and wherein the growth of the tumor is inhibited for at least 10, 20, 30, 40, 50, 60, 70, 80, 90, 100, 120, 150, 200, 240 days.

3. A method of treating a solid tumor with enhanced efficacy in a subject in need thereof, the method comprising administering to the subject a pharmaceutical composition comprising a population of immune cells comprising a mutated endogenous CD5 gene and a heterologous chimeric antigen receptor (CAR), wherein the CAR comprises an antigen binding domain, a transmembrane domain, and an intracellular domain, wherein the antigen binding domain binds to a solid tumor antigen, and wherein the efficacy of the population of immune cells comprising the mutated endogenous CD5 gene is greater than a population of immune cells comprising a non-mutated endogenous CD5 gene.

4. The method of claim 1, wherein the pharmaceutical composition comprises about 1×10⁶ to about 1×10⁹, about 1×10⁷ to about 1×10⁹, about 1×10⁸ to about 1×10⁹, about 2×10⁸ to about 1×10⁹, about 3×10⁸ to about 1×10⁹, about 3×10⁸ to about 1×10⁹, about 3×10⁸ to about 1×10⁹, about 4×10⁸ to about 1×10⁹, about 5×10⁸ to about 1×10⁹, about 6×10⁸, about 7×10⁸ to about 1×10⁹, about 8×10⁸, about 9×10⁸ to about 1×10⁹ of the immune cells.

5. The method of claim 1, wherein at least 75%, 80%, 85%, 90%, 91%, 92%, 93%, 94%, 95%, 96%, 97%, 98%, 99%, or 100% of the population of immune cells comprise the mutated endogenous CD5 gene.

6. The method of claim 1, wherein at least 50%, 55%, 60%, 65%, 75%, 80%, 85%, 90%, 91%, 92%, 93%, 94%, 95%, 96%, 97%, 98%, 99%, or 100% of the population of immune cells comprise the heterologous chimeric antigen receptor (CAR).

7. The method of claim 1, wherein the immune cells do not express or comprise an endogenous full length CD5 protein.

8. The method of claim 1, wherein the mutated CD5 gene has an exon 1 or exon 2 mutation, insertion or deletion.

9. The method of claim 8, wherein the exon 1 or exon 2 mutation, insertion or deletion inhibits or reduces the expression of the endogenous full length CD5 protein.

10. The method of claim 1, wherein the endogenous CD5 gene comprises a gene edited mutation, insertion or deletion.

11. The method of claim 10, wherein the gene edited mutation, insertion or deletion is a nuclease mediated gene mutation, insertion or deletion.

12. The method of claim 11, wherein the nuclease mediated gene mutation, insertion or deletion targets exon 1 or exon 2 of the CD5 gene.

13. The method of claim 1, wherein the gene edited or nuclease mediated gene mutation, insertion or deletion utilizes an sgRNA comprising the nucleotide sequence of SEQ ID NO: 45.

14. The method of claim 1, wherein the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), 0-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

15. The method of claim 1, wherein the modified immune cell is a T cell.

16. The method of claim 1, wherein the antigen binding domain of the CAR comprises a complementarity determining region (CDR) comprising an amino acid sequence selected from the group consisting of SEQ ID NOs: 27-32 and 39-44.

17. The method of claim 1, wherein the antigen binding domain of the CAR comprises a heavy chain variable region comprising the amino acid sequence selected from the group consisting of SEQ ID NOs: 19, 25, and 37and/or a light chain variable region comprising the amino acid sequence selected from the group consisting of SEQ ID NOs: 20, 26, and 38.

18. The method of claim 1, wherein the antigen binding domain of the CAR comprises an scFv comprising the amino acid sequence selected from the group consisting of SEQ ID NOs: 17, 18, 23, 24, 35, or 36.

19. The method of claim 1, wherein the CAR comprises an amino acid sequence selected from the group consisting of SEQ ID NOs: 15, 16, 21, 22, 33, and 34.

20. The method of claim 1, wherein the CAR is encoded by a nucleic acid sequence selected from the group consisting of SEQ ID NOs: 1-6.

21. The method of claim 1, wherein the solid tumor is prostate cancer, pancreatic ductal adenocarcinoma (“PDAC”), fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

22. A method of treating a solid tumor in a subject in need thereof, the method comprising:
administering to the subject a vector comprising a targeting moiety that binds to a CD5 expressing immune cell and a polynucleotide encoding a gene editing system and a chimeric antigen receptor (CAR),
wherein the CAR comprises an antigen binding domain, a transmembrane domain, and an intracellular domain, wherein the antigen binding domain binds to a solid tumor antigen,
wherein the gene editing system is configured to modify the endogenous CD5 gene to inhibit, eliminate, or reduce expression of endogenous CD5, and
wherein the vector transduces a CD5 expressing immune cell in the subject to mutate the endogenous CD5 gene to inhibit or reduce expression of endogenous CD5 and express the CAR in the immune cell with the mutated endogenous CD5 gene.

23. The method of claim 22, wherein the vector is a viral vector.

24. The method of claim 23, wherein the viral vector is a lentivirus.

25. The method of claim 22, wherein the targeting moiety is an antibody or other type of targeting moiety.

26. The method of claim 22, wherein the solid tumor is fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases), carcinomas, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, sarcomas fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, or other soft tissue sarcomas.

27. The method of claim 22, wherein the transduced cells do not express or comprise an endogenous full length CD5 protein.

28. The method of claim 22, wherein the mutated CD5 gene has an exon 1 or exon 2 mutation, insertion or deletion.

29. The method of claim 28, wherein the exon 1 or exon 2 mutation, insertion or deletion inhibits or reduces the expression of the endogenous full length CD5 protein.

30. The method of claim 22, wherein the endogenous CD5 gene comprises a gene edited gene mutation, insertion or deletion.

31. The method of claim 30, wherein the gene edited gene mutation, insertion or deletion is a CRISPR/Cas9 mediated gene mutation, insertion or deletion.

32. The method of claim 30, wherein the gene edited gene mutation, insertion or deletion targets exon 1 or exon 2 of the CD5 gene.

33. The method of claim 32, wherein the gene edited gene mutation, insertion or deletion utilizes an sgRNA comprising the nucleotide sequence of SEQ ID NO: 45.

34. The method of claim 22, wherein the antigen binding domain of the CAR is capable of binding an antigen selected from the group consisting of mesothelin, CD5, CD19, CD2, CD7, a tumor-specific antigen (TSA), a tumor associated antigen (TAA), a glioma-associated antigen, carcinoembryonic antigen (CEA), 0-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-i/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, pi85erbB2, pi80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

35. The method of claim 22, wherein the modified immune cell is a T cell.

36. The method of claim 22, wherein the antigen binding domain of the CAR comprises a complementarity determining region (CDR) comprising an amino acid sequence selected from the group consisting of SEQ ID NOs: 27-32 and 39-44.

37. The method of claim 22, wherein the antigen binding domain of the CAR comprises a heavy chain variable region comprising the amino acid sequence selected from the group consisting of SEQ ID NOs: 19, 25, and 37 and/or a light chain variable region comprising the amino acid sequence selected from the group consisting of SEQ ID NOs: 20, 26, and 38.

38. The method of claim 22, wherein the antigen binding domain of the CAR comprises an scFv comprising the amino acid sequence selected from the group consisting of SEQ ID NOs: 17, 18, 23, 24, 35, or 36.

39. The method of claim 22, wherein the CAR comprises an amino acid sequence selected from the group consisting of SEQ ID NOs: 15, 16, 21, 22, 33, and 34.

40. The method of claim 22, wherein the CAR is encoded by a nucleic acid sequence selected from the group consisting of SEQ ID NOs: 1-6.

41. A modified immune cell comprising a chimeric antigen receptor (CAR), wherein the CAR comprises an antigen binding domain, a transmembrane domain, and an intracellular domain, and wherein the endogenous CD5 gene has been mutated, modified, disrupted, or knocked-out, and wherein the antigen binding domain binds to a solid tumor antigen.

42. The modified immune cell of claim 41, wherein the antigen binding domain of the CAR binds to mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

43. A composition comprising a population of the modified immune cells of claim 41.

44. The composition of claim 43, wherein at least 75%, 80%, 85%, 90%, 91%, 92%, 93%, 94%, 95%, 96%, 97%, 98%, 99%, or 100% of the population of the modified immune cells comprise the mutated endogenous CD5 gene.

45. The composition of claim 43, wherein at least 50%, 55%, 60%, 65%, 75%, 80%, 85%, 90%, 91%, 92%, 93%, 94%, 95%, 96%, 97%, 98%, 99%, or 100% of the population of immune cells comprise the heterologous chimeric antigen receptor (CAR).

46. A method of generating the modified immune cell of claim 41, the method comprising transfecting or transducing the immune cell with a nucleic acid encoding the CAR and/or at least one nucleic acid that knocks-out, mutates, or disrupts endogenous CD5 gene, such as an exon of the CD5 gene, including but not limited to exon 1 or exon 2 of the CD5 gene, wherein the CAR comprises an antigen binding domain, a transmembrane domain, and an intracellular domain, wherein the antigen binding domain binds to a solid tumor antigen.

47. The method of claim 46, wherein the antigen binding domain binds to mesothelin, CD5, CD19, CD2, CD7, a tumor-specific antigen (TSA), a tumor associated antigen (TAA), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her1, Her2/neu, survivin, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, MART-1/MelanA (MART-I), gp100 (Pmel 17), tyrosinase, TRP-1, TRP-2, MAGE-1, MAGE-3, BAGE, GAGE-1, GAGE-2, p15, Ras, BCR-ABL, E2A-PRL, H4-RET, IGH-IGK, MYL-RAR, EBVA, HPV antigen E6, HPV antigen E7, TSP-180, MAGE-4, MAGE-5, MAGE-6, RAGE, NY-ESO, pi85erbB2, pi80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

48. The method of claim 46, wherein the immune cell is transduced in vivo or ex vivo.

49. The method of claim 48, wherein the immune cell is transduced in vivo.

50. The method of claim 46, wherein the immune cell is a T cell.


SEQUENCE LISTING (77 sequences)

SEQ ID NO: 1 - DNA, 738 bp, synthetic construct
  source 1..738: mol_type = other DNA
ggatccaacattgtactgacgcaaagcccctcatctttgtctgagtcactcggcggcaaagtaaccatcacatgcaaggccagtcaagacatcaataaatatattgcttggtatcagtataaacccggcaaggggccgcgactgctgattcactacacgagtaccttgcaaccgggcattccgagccgatttagtggcagtggctcaggtcgcgattactcattctcaataagtaatctcgaaccggaagacatagctacttattattgcttgcagtacgataatttgtggaccttcgggggtggtacaaagttggaaataaagggtggcggagggagcggcggtggaggaagcggaggcggaggttccgaggtccaactcgtagaatcaggtcccggattggtgcaaccatcccagagcctctctattacatgcacggtctctggatttagtctgaccaattacgatgtgcattgggtgcgccagtctcccggcaaggggttggaatggcttggcgttatatggaactacggaaatacagactataacgccgcgtttatctctcggctgagtatacggaaagacagtagtaaatcccaggtcttttttacgatgtcatccctgcaaacgccagataccgcaatatattactgcgccaggaaccacggtgatggttattataattggtacttcgatgtgtggggtactggcactacagtcacagtatcttcatctaga

SEQ ID NO: 2 - DNA, 741 bp, synthetic construct
  source 1..741: mol_type = other DNA
ggatcccaggtccagctgaaagaaagcggtccagagctggaaaaacccggtgcgagcgtcaaaatatcatgtaaagcaagcgggtattcattcaccgcgtactctatgaactgggttaagcaaaacaacggtatgtccttggagtggatagggtctatcgacccgtattatggggacacaaaatacgcgcagaaattcaaggggaaggccaccctgaccgtagataaagctagttctactgcgtacttgcaactgaaaagcctcacttctgaggactctgccgtctactactgtgctcggcgaatgataacgacgggggactggtatttcgatgtttggggtacagggactacggtgactgtcagtagcggtggcggagggagcggcggtggaggaagcggaggcggaggttcccatatcgtcttgactcaatcacctagttctttgtctgcgtcccttggcgaccgagtcaccatatcttgcagagcgtcacaggacatttcaacgtacctcaactggtatcagcaaaaaccggacgggactgtcaagctcttgatcttctacacttccagactccacgccggggtgccaagcagatttagtggctctggcagcgggacacaccatagtcttacaatcagcaatcttgagcaagaagacatagccacgtatttctgccagcaaggtaactcacttccgttcacgtttggtagtggcaccaaactggagataaaatccgga

SEQ ID NO: 3 - DNA, 741 bp, synthetic construct
  source 1..741: mol_type = other DNA
ggatcccatatcgtcttgactcaatcacctagttctttgtctgcgtcccttggcgaccgagtcaccatatcttgcagagcgtcacaggacatttcaacgtacctcaactggtatcagcaaaaaccggacgggactgtcaagctcttgatcttctacacttccagactccacgccggggtgccaagcagatttagtggctctggcagcgggacacaccatagtcttacaatcagcaatcttgagcaagaagacatagccacgtatttctgccagcaaggtaactcacttccgttcacgtttggtagtggcaccaaactggagataaaaggtggcggagggagcggcggtggaggaagcggaggcggaggttcccaggtccagctgaaagaaagcggtccagagctggaaaaacccggtgcgagcgtcaaaatatcatgtaaagcaagcgggtattcattcaccgcgtactctatgaactgggttaagcaaaacaacggtatgtccttggagtggatagggtctatcgacccgtattatggggacacaaaatacgcgcagaaattcaaggggaaggccaccctgaccgtagataaagctagttctactgcgtacttgcaactgaaaagcctcacttctgaggactctgccgtctactactgtgctcggcgaatgataacgacgggggactggtatttcgatgtttggggtacagggactacggtgactgtcagtagctccgga

SEQ ID NO: 4 - DNA, 732 bp, synthetic construct
  source 1..732: mol_type = other DNA
ggatccgaggttaaactcgtggagagcggtgccgaactcgtccgaagtggtgcttccgttaaactcagttgtgccgcgtcaggatttaacataaaagattactacattcactgggtcaaacagcgcccggagcaggggcttgaatggatcgggtggattgatcctgaaaacgggcgcaccgaatatgctcccaagttccagggcaaagctactatgaccgctgacacctctagtaacactgcctacctgcagttgagctctcttacgtctgaggataccgctgtgtactactgtaataacggaaattatgtacgacactattacttcgactactgggggcagggcactactgtgactgtatctagcggtggcggagggagcggcggtggaggaagcggaggcggaggttccgattggctcacacaatcccctgcaatcctgagtgcatctccaggcgagaaagtaactatgacttgcagagctataagctctgtgtcctacatgcactggtatcagcagaagccaggttcttccccgaagccgtggatatatgctacaagcaatttggcatccggtgttcccgcccggtttagtggctccggttctgggacaagttactccctcacgatcagcagggttgaagccgaggacgctgccacttactattgccaacagtggtcaagtaaccccaggactttcgggggaggaactaaacttgaaatcaaatctaga

SEQ ID NO: 5 - DNA, 732 bp, synthetic construct
  source 1..732: mol_type = other DNA
ggatccgattggctcacacaatcccctgcaatcctgagtgcatctccaggcgagaaagtaactatgacttgcagagctataagctctgtgtcctacatgcactggtatcagcagaagccaggttcttccccgaagccgtggatatatgctacaagcaatttggcatccggtgttcccgcccggtttagtggctccggttctgggacaagttactccctcacgatcagcagggttgaagccgaggacgctgccacttactattgccaacagtggtcaagtaaccccaggactttcgggggaggaactaaacttgaaatcaaaggtggcggagggagcggcggtggaggaagcggaggcggaggttccgaggttaaactcgtggagagcggtgccgaactcgtccgaagtggtgcttccgttaaactcagttgtgccgcgtcaggatttaacataaaagattactacattcactgggtcaaacagcgcccggagcaggggcttgaatggatcgggtggattgatcctgaaaacgggcgcaccgaatatgctcccaagttccagggcaaagctactatgaccgctgacacctctagtaacactgcctacctgcagttgagctctcttacgtctgaggataccgctgtgtactactgtaataacggaaattatgtacgacactattacttcgactactgggggcagggcactactgtgactgtatctagctctaga

SEQ ID NO: 6 - DNA, 738 bp, synthetic construct
  source 1..738: mol_type = other DNA
ggatccgaggtccaactcgtagaatcaggtcccggattggtgcaaccatcccagagcctctctattacatgcacggtctctggatttagtctgaccaattacgatgtgcattgggtgcgccagtctcccggcaaggggttggaatggcttggcgttatatggaactacggaaatacagactataacgccgcgtttatctctcggctgagtatacggaaagacagtagtaaatcccaggtcttttttacgatgtcatccctgcaaacgccagataccgcaatatattactgcgccaggaaccacggtgatggttattataattggtacttcgatgtgtggggtactggcactacagtcacagtatcttcaggtggcggagggagcggcggtggaggaagcggaggcggaggttccaacattgtactgacgcaaagcccctcatctttgtctgagtcactcggcggcaaagtaaccatcacatgcaaggccagtcaagacatcaataaatatattgcttggtatcagtataaacccggcaaggggccgcgactgctgattcactacacgagtaccttgcaaccgggcattccgagccgatttagtggcagtggctcaggtcgcgattactcattctcaataagtaatctcgaaccggaagacatagctacttattattgcttgcagtacgataatttgtggaccttcgggggtggtacaaagttggaaataaagtctaga

SEQ ID NO: 7 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 8 - protein, 24 aa, synthetic construct
IYIWAPLAGTCGVLLLSLVITLYC

SEQ ID NO: 9 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 10 - protein, 45 aa, synthetic construct
TTTPAPRPPTPAPTIASQPLSLRPEACRPAAGGAVHTRGLDFACD

SEQ ID NO: 11 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 12 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 13 - protein, 42 aa, synthetic construct
KRGRKKLLYIFKQPFMRPVQTTQEEDGCSCRFPEEEEGGCEL

SEQ ID NO: 14 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 494 aa, synthetic construct
MALPVTALLLPLALLLHAARPGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKGGGGSGGGGSGGGGSEVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 494 aa, synthetic construct
MALPVTALLLPLALLLHAARPGSEVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSGGGGSGGGGSGGGGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 242 aa, synthetic construct
NIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIKGGGGSGGGGSGGGGSEVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSS

SEQ ID NO: 18 - protein, 242 aa, synthetic construct
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSSGGGGSGGGGSGGGGSNIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIK

SEQ ID NO: 19 - protein, 121 aa, synthetic construct
EVQLVESGPGLVQPSQSLSITCTVSGFSLTNYDVHWVRQSPGKGLEWLGVIWNYGNTDYNAAFISRLSIRKDSSKSQVFFTMSSLQTPDTAIYYCARNHGDGYYNWYFDVWGTGTTVTVSS

SEQ ID NO: 20 - protein, 106 aa, synthetic construct
NIVLTQSPSSLSESLGGKVTITCKASQDINKYIAWYQYKPGKGPRLLIHYTSTLQPGIPSRFSGSGSGRDYSFSISNLEPEDIATYYCLQYDNLWTFGGGTKLEIK

SEQ ID NO: 21 - protein, 491 aa, synthetic construct
MALPVTALLLPLALLLHAARPGSQVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDVWGTGTTVTVSSGGGGSGGGGSGGGGSHIVLTQSPSSLSASLGDRVTISCRASQDISTYLNWYQQKPDGTVKLLIFYTSRLHAGVPSRFSGSGSGTHHSLTISNLEQEDIATYFCQQGNSLPFTFGSGTKLEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 491 aa, synthetic construct
MALPVTALLLPLALLLHAARPGSHIVLTQSPSSLSASLGDRVTISCRASQDISTYLNWYQQKPDGTVKLLIFYTSRLHAGVPSRFSGSGSGTHHSLTISNLEQEDIATYFCQQGNSLPFTFGSGTKLEIKGGGGSGGGGSGGGGSQVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDVWGTGTTVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 247 aa, synthetic construct
GSQVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDVWGTGTTVTVSSGGGGSGGGGSGGGGSHIVLTQSPSSLSASLGDRVTISCRASQDISTYLNWYQQKPDGTVKLLIFYTSRLHAGVPSRFSGSGSGTHHSLTISNLEQEDIATYFCQQGNSLPFTFGSGTKLEIKSG

SEQ ID NO: 24 - protein, 243 aa, synthetic construct
HIVLTQSPSSLSASLGDRVTISCRASQDISTYLNWYQQKPDGTVKLLIFYTSRLHAGVPSRFSGSGSGTHHSLTISNLEQEDIATYFCQQGNSLPFTFGSGTKLEIKGGGGSGGGGSGGGGSQVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDVWGTGTTVTVSS

SEQ ID NO: 25 - protein, 121 aa, synthetic construct
QVQLKESGPELEKPGASVKISCKASGYSFTAYSMNWVKQNNGMSLEWIGSIDPYYGDTKYAQKFKGKATLTVDKASSTAYLQLKSLTSEDSAVYYCARRMITTGDWYFDVWGTGTTVTVSS

SEQ ID NO: 26 - protein, 107 aa, synthetic construct
HIVLTQSPSSLSASLGDRVTISCRASQDISTYLNWYQQKPDGTVKLLIFYTSRLHAGVPSRFSGSGSGTHHSLTISNLEQEDIATYFCQQGNSLPFTFGSGTKLEIK

SEQ ID NO: 27 - protein, 5 aa, synthetic construct
AYSMN

SEQ ID NO: 28 - protein, 17 aa, synthetic construct
SIDPYYGDTKYAQKFKG

SEQ ID NO: 29 - protein, 12 aa, synthetic construct
RMITTGDWYFDV

SEQ ID NO: 30 - protein, 11 aa, synthetic construct
RASQDISTYLN

SEQ ID NO: 31 - protein, 7 aa, synthetic construct
YTSRLHA

SEQ ID NO: 32 - protein, 9 aa, synthetic construct
QQGNSLPFT

SEQ ID NO: 33 - protein, 492 aa, synthetic construct
MALPVTALLLPLALLLHAARPGSEVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDYWGQGTTVTVSSGGGGSGGGGSGGGGSDWLTQSPAILSASPGEKVTMTCRAISSVSYMHWYQQKPGSSPKPWIYATSNLASGVPARFSGSGSGTSYSLTISRVEAEDAATYYCQQWSSNPRTFGGGTKLEIKSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 492 aa, synthetic construct
MALPVTALLLPLALLLHAARPGSDWLTQSPAILSASPGEKVTMTCRAISSVSYMHWYQQKPGSSPKPWIYATSNLASGVPARFSGSGSGTSYSLTISRVEAEDAATYYCQQWSSNPRTFGGGTKLEIKGGGGSGGGGSGGGGSEVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDYWGQGTTVTVSSSRTTTPAPRPPTPAPTIASQPLSLRPEACRPAAGGAVHTRGLDFACDIYIWAPLAGTCGVLLLSLVITLYCHMKRGRKKLLYIFKQPFMRPVQTTQEEDGCSCRFPEEEEGGCELTSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 240 aa, synthetic construct
EVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDYWGQGTTVTVSSGGGGSGGGGSGGGGSDWLTQSPAILSASPGEKVTMTCRAISSVSYMHWYQQKPGSSPKPWIYATSNLASGVPARFSGSGSGTSYSLTISRVEAEDAATYYCQQWSSNPRTFGGGTKLEIK

SEQ ID NO: 36 - protein, 240 aa, synthetic construct
DWLTQSPAILSASPGEKVTMTCRAISSVSYMHWYQQKPGSSPKPWIYATSNLASGVPARFSGSGSGTSYSLTISRVEAEDAATYYCQQWSSNPRTFGGGTKLEIKGGGGSGGGGSGGGGSEVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDYWGQGTTVTVSS

SEQ ID NO: 37 - protein, 120 aa, synthetic construct
EVKLVESGAELVRSGASVKLSCAASGFNIKDYYIHWVKQRPEQGLEWIGWIDPENGRTEYAPKFQGKATMTADTSSNTAYLQLSSLTSEDTAVYYCNNGNYVRHYYFDYWGQGTTVTVSS

SEQ ID NO: 38 - protein, 107 aa, synthetic construct
DWLTQSPAILSASPGEKVTMTCRAISSVSYMHWYQQKPGSSPKPWIYATSNLASGVPARFSGSGSGTSYSLTISRVEAEDAATYYCQQWSSNPRTFGGGTKLEIKSR

SEQ ID NO: 39 - protein, 5 aa, synthetic construct
DYYIH

SEQ ID NO: 40 - protein, 17 aa, synthetic construct
WIDPENGRTEYAPKFQG

SEQ ID NO: 41 - protein, 11 aa, synthetic construct
GNYVRHYYFDY

SEQ ID NO: 42 - protein, 10 aa, synthetic construct
RAISSVSYMH

SEQ ID NO: 43 - protein, 7 aa, synthetic construct
ATSNLAS

SEQ ID NO: 44 - protein, 9 aa, synthetic construct
QQWSSNPRT

SEQ ID NO: 45 - DNA, 20 bp, synthetic construct
  source 1..20: mol_type = other DNA
cggctcagctggtatgaccc

SEQ ID NO: 46 - protein, 5 aa, synthetic construct
  REPEAT 1..5: note = repeat n times, where n represents an integer of at least 1
GSGGS

SEQ ID NO: 47 - protein, 4 aa, synthetic construct
  REPEAT 1..4: note = repeat n times, where n represents an integer of at least 1
GGGS

SEQ ID NO: 48 - protein, 5 aa, synthetic construct
  REPEAT 1..5: note = repeat n times, where n represents an integer of at least 1
GGGGS

SEQ ID NO: 49 - protein, 4 aa, synthetic construct
GGSG

SEQ ID NO: 50 - protein, 5 aa, synthetic construct
GGSGG

SEQ ID NO: 51 - protein, 5 aa, synthetic construct
GSGSG

SEQ ID NO: 52 - protein, 5 aa, synthetic construct
GSGGG

SEQ ID NO: 53 - protein, 5 aa, synthetic construct
GGGSG

SEQ ID NO: 54 - protein, 5 aa, synthetic construct
GSSSG

SEQ ID NO: 55 - protein, 5 aa, synthetic construct
GGGGS

SEQ ID NO: 56 - protein, 15 aa, synthetic construct
GGGGSGGGGSGGGGS

SEQ ID NO: 57 - DNA, 45 bp, synthetic construct
  source 1..45: mol_type = other DNA
ggtggcggtggctcgggcggtggtgggtcgggtggcggcggatct

SEQ ID NO: 58 - protein, 5 aa, synthetic construct
DKTHT

SEQ ID NO: 59 - protein, 4 aa, synthetic construct
CPPC

SEQ ID NO: 60 - protein, 15 aa, synthetic construct
CPEPKSCDTPPPCPR

SEQ ID NO: 61 - protein, 12 aa, synthetic construct
ELKTPLGDTTHT

SEQ ID NO: 62 - protein, 10 aa, synthetic construct
KSCDKTHTCP

SEQ ID NO: 63 - protein, 7 aa, synthetic construct
KCCVDCP

SEQ ID NO: 64 - protein, 7 aa, synthetic construct
KYGPPCP

SEQ ID NO: 65 - protein, 15 aa, synthetic construct
EPKSCDKTHTCPPCP

SEQ ID NO: 66 - protein, 12 aa, synthetic construct
ERKCCVECPPCP

SEQ ID NO: 67 - protein, 17 aa, synthetic construct
ELKTPLGDTTHTCPRCP

SEQ ID NO: 68 - protein, 12 aa, synthetic construct
SPNMVPHAHHAQ

SEQ ID NO: 69 - protein, 15 aa, synthetic construct
EPKSCDKTYTCPPCP

SEQ ID NO: 70 - DNA, 20 bp, synthetic construct
  source 1..20: mol_type = other DNA
ctctctgagagcgagatacc

SEQ ID NO: 71 - DNA, 20 bp, synthetic construct
  source 1..20: mol_type = other DNA
cgggtatctcgctctcagag

SEQ ID NO: 72 - DNA, 20 bp, synthetic construct
  source 1..20: mol_type = other DNA
cataccagctgagccgtccg

SEQ ID NO: 73 - DNA, 20 bp, synthetic construct
  source 1..20: mol_type = other DNA
cggctcagctggtatgaccc

SEQ ID NO: 74 - DNA, 20 bp, synthetic construct
  source 1..20: mol_type = other DNA
gcagacttttgacgcttgac

SEQ ID NO: 75 - DNA, 20 bp, synthetic construct
  source 1..20: mol_type = other DNA
aatcatctgctacggacaac

SEQ ID NO: 76 - DNA, 20 bp, synthetic construct
  source 1..20: mol_type = other DNA
ggcgggggccttgtcgttgg

SEQ ID NO: 77 - DNA, 20 bp, synthetic construct
  source 1..20: mol_type = other DNA
agacaacacctccaacgaca